(19) United States
(12) Patent Application Publication
(10) Pub. No.: US 2025/0228669 A1
(43) Pub. Date: Jul. 17, 2025

(54) DYNAMIC VENOUS OCCLUSION DEVICES

(71) Applicant: EDWARDS LIFESCIENCES CORPORATION, Irvine, CA (US)

(72) Inventors: Brandon Sina Dehdashtian, Irvine, CA (US); Alexander H. Siemons, Yorba Linda, CA (US); Zachary Christopher Berwick, Tustin, CA (US); Allen Jeong Keel, Irvine, CA (US); Mazen Saadi Albaghdadi, Irvine, CA (US); Yaeer E. Lev, Torrance, CA (US); Atiya Makhdoom Ahmad, Irvine, CA (US); Gustavo Rios, Pasadena, CA (US); Farzad Azimpour, Irvine, CA (US); Anthony Ciro Vrba, San Clemente, CA (US); Arvin T. Chang, Yorba Linda, CA (US); Omar Fawzi Azanki, Coto de Caza, CA (US); Emil Karapetian, Huntington Beach, CA (US)

(73) Assignee: EDWARDS LIFESCIENCES CORPORATION, Irvine, CA (US)

(21) Appl. No.: 19/169,876

(22) Filed: Apr. 3, 2025

Related U.S. Application Data
(63) Continuation of application No. PCT/US2023/076297, filed on Oct. 6, 2023.
(60) Provisional application No. 63/378,794, filed on Oct. 7, 2022.

(51) Int. Cl. A61F 2/24

(52) U.S. Cl. CPC A61F 2/2475; A61F 2250/0003

(57) ABSTRACT

A system for regulating blood flow through a blood vessel of a heart includes a valve and a controller. The valve includes an outer frame and two or more leaflets and is sized for placement at least partially within the blood vessel. The two or more leaflets are sized to overlap or contact each other in an expanded state. The controller is disposed outside the heart and transmits signals to the valve.

### RELATED APPLICATION

This application is a continuation of International Patent Application No. PCT/PCT/US2023/076297, filed Oct. 6, 2023, which claims the benefit of U.S. Provisional Application No. 63/378,794, filed Oct. 7, 2022, the disclosures of which are hereby incorporated by reference in their entirety.

### BACKGROUND

The present invention relates generally to the field of medical devices and procedures.

Redistribution of blood from the splanchnic venous circulation to the inferior vena cava (IVC) can contribute to increases in central venous pressure (CVP), pulmonary artery pressure, and/or pulmonary capillary wedge pressure (PCWP), particularly during periods of elevated sympathetic tone (e.g., exercise) in heart failure patients.

### SUMMARY

Described herein are one or more methods and/or devices to facilitate management of blood flow through and/or into one or more blood vessels and/or chambers of a heart.

For purposes of summarizing the disclosure, certain aspects, advantages, and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 provides a schematic representation of portions of the renal circulation.

FIG. 2 provides another schematic representation of the splanchnic circulation, illustrating blood flow from the aorta to the inferior vena cava (IVC).

FIG. 3 illustrates portions of the splanchnic venous circulation acting as a blood reservoir between the aorta and the IVC.

FIG. 4 illustrates an example system for modulating blood flow through one or more blood vessels, which can include the IVC and/or SVC of a heart.

FIG. 5 illustrates an example occlusion valve configured to dynamically occlude one or more blood vessels of a heart in accordance with one or more examples.

FIG. 6A-6C provide overhead views of an example occlusion valve configured to dynamically occlude one or more blood vessels of a heart in accordance with one or more examples.

FIGS. 7A-7C provide overhead views of an example occlusion valve configured to dynamically occlude one or more blood vessels of a heart in accordance with one or more examples.

FIGS. 8A and 8B illustrate another example occlusion valve configured to dynamically occlude one or more blood vessels of a heart in accordance with one or more examples.

FIGS. 9A-9C illustrate another example occlusion valve configured to dynamically occlude one or more blood vessels of a heart in accordance with one or more examples.

FIG. 10 illustrates an example system comprising one or more valves in accordance with one or more examples described herein.

FIG. 11 illustrates an example frame of an occlusion valve described in any of the examples herein.

FIG. 12 provides an overhead view of a valve comprising multiple leaflets 1205 in accordance with one or more examples described herein.

FIG. 13 provides an overhead view of a valve comprising multiple leaflets in accordance with one or more examples described herein.

FIG. 14 provides a side view of a valve comprising multiple leaflets extending from a frame in accordance with one or more examples described herein.

FIGS. 15A-15C illustrate an example valve and/or one or more components of a valve in accordance with one or more examples described herein.

FIGS. 16A and 16B illustrate an example occlusion valve, which may comprise various component of valves described herein in accordance with one or more examples.

FIGS. 17A-17D illustrate an example valve configured to dynamically occlude one or more blood vessels of a heart in accordance with one or more examples herein.

FIGS. 18A and 18B illustrate an example valve configured to dynamically occlude one or more blood vessels of a heart in accordance with one or more examples herein.

FIG. 19 illustrates an example circuit configured to control and/or power one or more valves described in examples herein.

FIG. 20 illustrates another circuit for controlling one or more valves described herein in accordance with one or more examples.

FIGS. 21A and 21B illustrate an example dynamic occlusion valve configured for placement at least partially within one or more blood vessels of a heart in accordance with one or more examples herein.

FIGS. 22A and 22B illustrate another example valve configured to dynamically occlude one or more blood vessels of a heart in accordance with one or more examples.

FIGS. 23A-23C illustrate another example valve configured to dynamically occlude one or more blood vessels of a heart in accordance with one or more examples herein.

FIGS. 24A and 24B illustrate another example valve configured to dynamically occlude one or more blood vessels of a heart in accordance with one or more examples.

FIGS. 25A and 25B illustrate an example ratcheting mechanism configured to allow for dynamic adjustments of one or more components of various valves described herein.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

### Overview

The following includes a general description of human cardiac anatomy that is relevant to certain inventive features and examples disclosed herein and is included to provide context for certain aspects of the present disclosure.

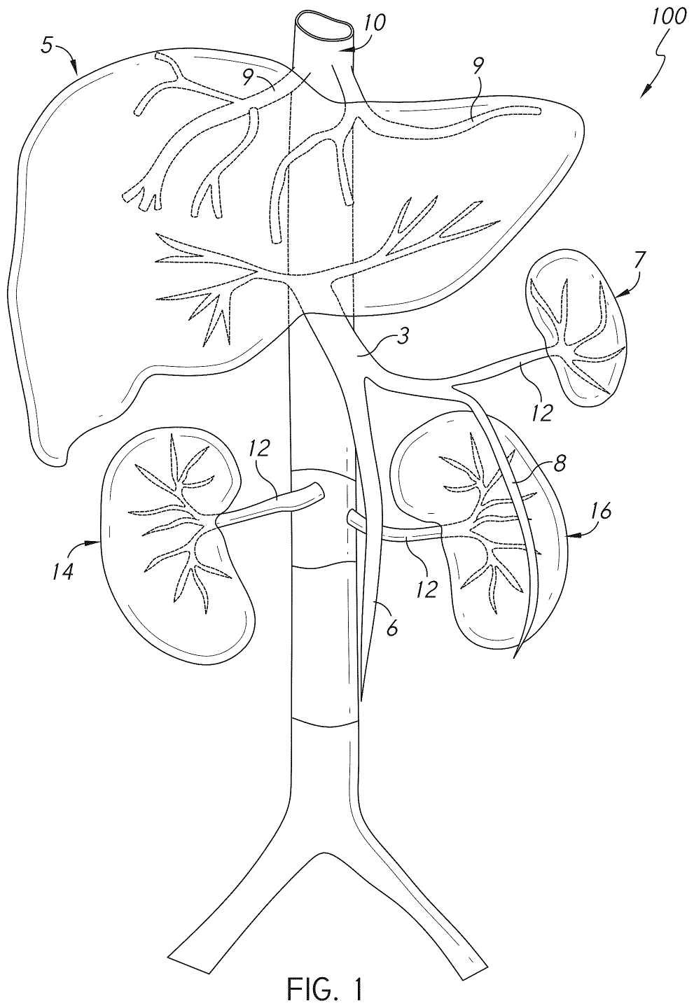

FIG. 1 provides a schematic representation of portions of the renal circulation 100 and/or splanchnic circulation. The term “splanchnic circulation” refers to blood flow originating from the celiac, superior mesenteric, and inferior mesenteric arteries to the abdominal gastrointestinal organs. The renal circulation 100 and/or splanchnic circulation receives approximately 25% of the cardiac output and holds a similar percentage of the total blood volume under normal conditions. The renal circulation 100 and/or splanchnic circulation can act as a site of cardiac output regulation and/or as a blood reservoir. Multiple regulatory pathways are involved in the distribution of the splanchnic circulation.

Total flow to the splanchnic viscera is controlled by resistance vessels in the mesenteric and hepatic arterial systems. The venous effluents from the splanchnic viscera converge to form the portal vein 3, which supplies approximately 75% of the total blood supply to the liver 5. The portal blood not only is high in substrate concentrations resulting from intestinal absorption but also tends to contain bacteria and endotoxin.

Renal veins 12 drain blood from the right kidney 14 and left kidney 16 and connect to the inferior vena cava 10 (IVC). The superior mesenteric vein 6 is a major venous tributary of the abdominal cavity that lies laterally to the superior mesenteric artery and serves to drain the vast majority of the organs of the abdominal cavity. The inferior mesenteric vein 8 drains blood from the large intestine. The splenic vein 12 is a blood vessel that drains blood from the spleen, the stomach fundus, and part of the pancreas.

The portal vein 3 receives blood from the stomach, intestines, pancreas, and spleen 7 and carries it into the liver 5 through the porta hepatis. The porta hepatis serves as the point of entry for the portal vein 3 and the proper hepatic artery, and is the point of exit for the bile passages.

Following processing of the blood by the liver 5, the blood collects in the central vein at the core of the lobule. Blood from these central veins ultimately converges in the right and left hepatic veins 9, which exit the superior surface of the liver 5 and empty into the IVC 10 to be distributed to the rest of the body.

The splanchnic venous circulation is highly compliant and can act as a blood reservoir that can be recruited in order to support the need for increased stressed blood volume during periods of elevated sympathetic tone, such as exertion, in order to support increased cardiac output and vasodilation of peripheral vessels supporting active muscles. However, heart failure patients can have multiple comorbidities that prevent them from using that additional blood volume. Such comorbidities can include chronotropic incompetence, inability to increase stroke volume, and/or peripheral microvascular dysfunction. This can lead to venous congestion and/or abrupt rises in pulmonary capillary wedge pressure (PCWP).

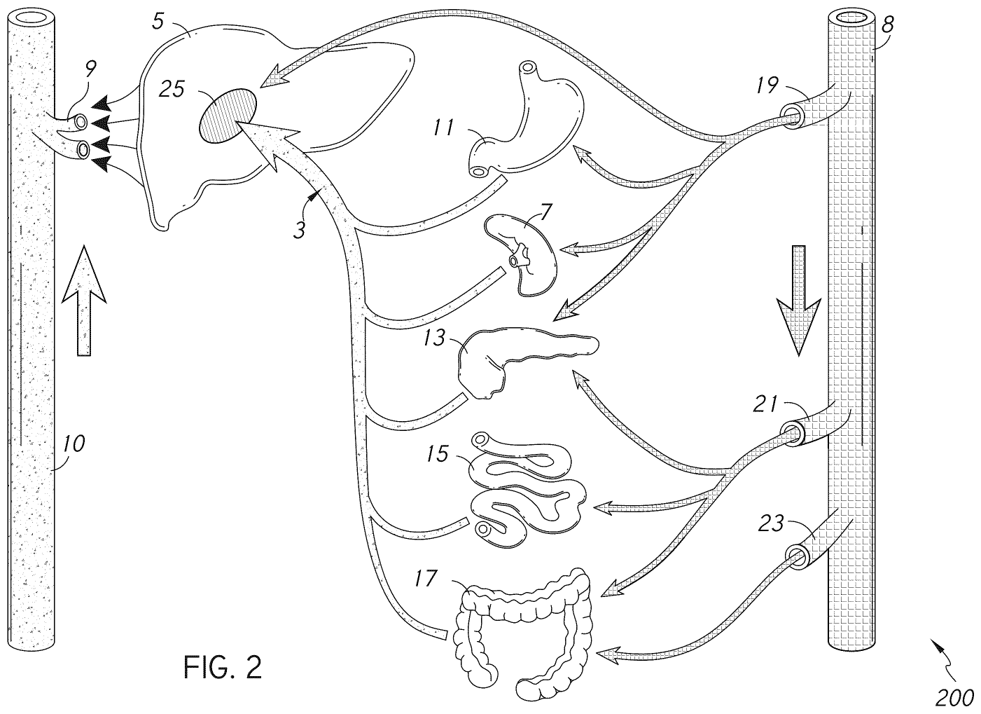

FIG. 2 provides another schematic representation of the splanchnic circulation 200, illustrating blood flow from the aorta 8 to the IVC 10. Blood travels from the aorta 8 to the abdominal gastrointestinal organs including the stomach 11, liver 5, spleen, 7, pancreas 13, small intestine 15, and large intestine 17. The splanchnic circulation 200 comprises three major branches of the abdominal aorta 9, including the coeliac artery 19, the superior mesenteric artery 21 (SMA), and the inferior mesenteric artery 23 (IMA). The hepatic portal circulation (e.g., the hepatic artery 18 and/or portal vein 3) delivers the majority of blood flow to the liver 5.

The coeliac artery 19 is the first major division of the abdominal aorta 8, branching at T12 in a horizontal direction ˜1.25 cm in length. It shows three main divisions such as the left gastric artery, common hepatic artery 18, and splenic artery and is the primary blood supply to the stomach 11, upper duodenum, spleen 7, and pancreas 13.

The SMA 21 arises from the abdominal aorta 8 anteriorly at L1, usually 1 cm inferior to the coeliac artery 19. The five major divisions of the SMA 21 are the inferior pancreaticoduodenal artery, intestinal arteries, ileocolic, right colic, and middle colic arteries. The SMA 21 supplies the lower part of the duodenum, jejunum, ileum, caecum, appendix, ascending colon, and two-thirds of the transverse colon. It is the largest of the splanchnic arterial vessels delivering >10% of the cardiac output and therefore has significant implications for embolic mesenteric ischaemia.

The IMA 23 branches anteriorly from the abdominal aorta 8 at L3, midway between the renal arteries and the iliac bifurcation. The main branches of the IMA 23 are the left colic artery, the sigmoid branches, and the superior rectal artery. It forms a watershed with the middle colic artery and supplies blood to the final third of the transverse colon, descending colon, and upper rectum.

Blood flow is conveyed into the liver 5 via the portal vein 3 into sinusoids 25 of the liver 5. The hepatic veins 9 convey the blood from the liver 5 to the IVC 10.

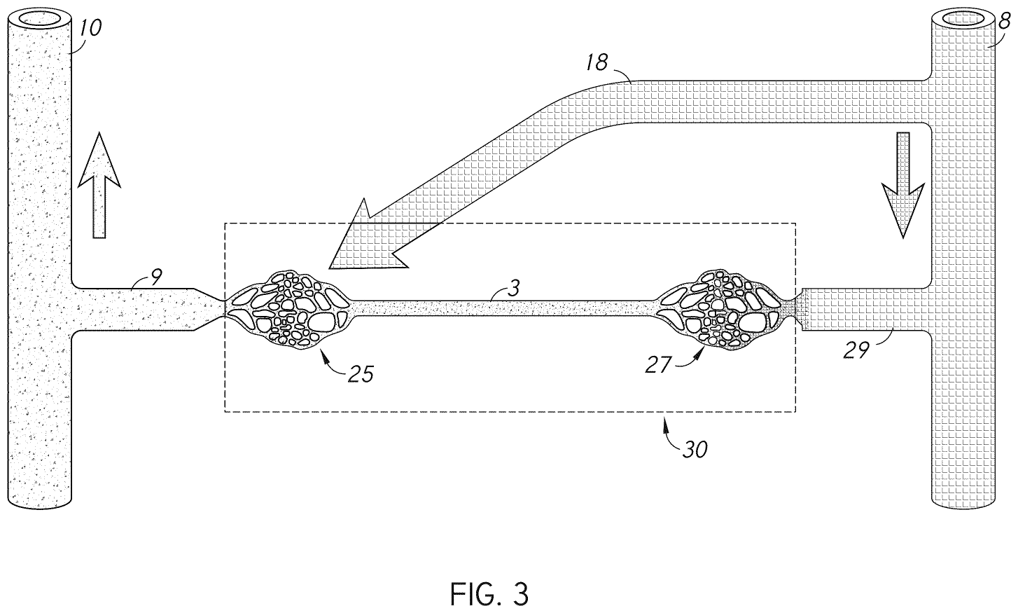

FIG. 3 illustrates portions of the splanchnic venous circulation 300 acting as a blood reservoir 30 between the aorta 8 and the IVC 10. The portal vein 30 conveys blood between the splanchnic organs 27 (e.g., the stomach, spleen, etc.) and the liver sinusoids 25. The liver sinusoids 25 also receive blood from the hepatic artery 18. The splanchnic organs 27 receive blood from the aorta 8 via various splanchnic arteries 29 (e.g., the SMA, IMA, etc.). The amount of blood contained in the portal vein 3 at any given time can be variable.

For some patients (especially patients experiencing heart failure) fluid redistribution from the splanchnic venous reservoir 30 to the IVC 10 and/or stressed blood volume can contribute to increases in central venous pressure (CVP), pulmonary artery pressure, and/or PCWP. This can be especially problematic during periods of elevated sympathetic tone, such as exertion, and/or can lead to pulmonary congestion that can impact a patient's quality of life and/or can lead to acute decompensation.

The splanchnic venous circulation 300, and particularly the portal vein 3, can advantageously provide a blood reserve to support the need for increased stressed blood volume during periods of elevated sympathetic tone. Because blood flow from the splanchnic venous circulation 300 is directed through the hepatic veins 9 and into the IVC 10, devices placed into the hepatic veins 9 and/or IVC 10 to limit blood flow can allow the reservoir 30 to expand with increased blood volume.

Examples described herein can relate to devices and/or methods that can advantageously limit, stagnate, and/or impede blood flow into the IVC 10 from the hepatic veins 9 to increase the pressure gradient between the IVC 10 and the liver and/or splanchnic venous circulation 300. In some examples, one or more flow-regulating implants may be configured for placement at least partially within the hepatic veins 9 and/or IVC 10 and/or at one or more junctions between the hepatic veins 9 and the IVC 10. As a result, blood flowing from the splanchnic venous reservoir 30 into the hepatic veins 9 can be slowed to increase blood volume in the splanchnic venous reservoir 30.

Some approaches to reducing volume redistribution can involve placing fixed orifice flow restrictors at or near the IVC 10. However, while restricting the flow from the hepatic veins 9 can be beneficial in preventing volume redistribution, too much restriction can cause hepatic congestion. It would therefore be advantageous to modulate the response and increase restriction only during volume redistribution.

Some examples presented herein relate to methods and/or devices for increasing the restriction of blood flow from the hepatic veins 9 and/or IVC 10 into the right atrium as pressures increase in the left atrium. The various devices can be implanted using a transcatheter transvenous approach, for example entering through the femoral vein. The delivery system can then be progressively unsheathed, and other components of the device can be sequentially implanted under fluoroscopic and echo guidance, if necessary.

Persons with chronic kidney disease (CKD) and heart failure suffer from reduced kidney function when pressure in the right atrium is high. Maintaining relatively low pressure in the right atrium allows the kidneys to more effectively filter blood. In certain conditions, surges of blood volume can lead to pressure build-up in the left atrium. Moreover, patients may accumulate blood volume in the venous system and at some point may increase the pressures in the right atrium and thus in the IVC as a result of volume status. This can be more likely in CKD patients that have weakened diuresis with nephron loss. Furthermore, increases in this pressure on the venous side of the kidneys may increase the likelihood of volume accumulation.

By controlling the return of blood, pressure build-up in the right atrium can be mitigated. Some examples described herein can assist in maintaining right atrium pressures relatively low when a surge in blood volume occurs.

The Superior Vena Cava (SVC) returns about 30% of the blood volume to the right atrium. Controlling the amount of blood return through the SVC can assist in mitigating pressure in the right atrium.

The solution presented herein relates to an implantable dynamic valve system that may be used in controlling pressure in veins returning to the heart, in particular the SVC. Some example dynamic valve systems can include, among other components, a valve that can be opened or closed to predetermined positions and latched, or held, in the predetermined position without constant power being supplied.

Acute heart failure is a common condition and can occur in patients who have long term heart failure. Such patients can have a sudden rapid onset of heart failure symptoms that cannot be treated using normal therapies. Patient symptoms can be rooted in volume overload. Increased volume in the circulatory system requires the heart to work harder to push blood through the body. Prolonged increased workload due to volume overload can wear out the heart, and can eventually result in symptoms of poor contractile function, shortness of breath (e.g., patient feeling like they are “drowning” due to backed up pressure in the lungs), and/or overall low blood volume circulation, among others.

Diuretics can provide a relatively less invasive solution for volume reduction, but may not always be effective. During episodes of acute heart failure, a patient may be admitted to an emergency room, where a stronger form of intravenous (IV) diuretics can be administered. If IV diuretics are not resulting in volume reduction, there may not be a reliable and effective therapy available. The cardiovascular community has discovered that although diuretics function as intended (e.g., giving the kidneys the ability to pull more water out of the circulatory system), the kidneys are often not able to pull water out of the blood due to low blood volume circulation since only partial amounts of blood passes through the kidneys. Moreover, patients suffering from compensated heart failure may experience insufficient forward pressure for kidney perfusion.

To increase blood volume passing through the kidneys, there must be a pressure gradient across the kidneys. To achieve this, there are two options; increase forward pressure (i.e., increase cardiac output) or decrease central venous pressure. Elevated central venous pressure has been determined to be the main contributor to the lack of blood flow through the kidneys and reduced diuretic efficacy.

Some examples presented herein provide devices and/or methods for reducing central venous pressure and/or workload on the right side of the heart (and in turn the left side of the heart) by decreasing the total volume in the central venous system via SVC or IVC occlusion therapy. In SVC occlusion therapy, the SVC ostia (where the SVC and right atrium meet) is temporarily occluded to restrict blood volume entering the right atrium/central venous system, which in turn reduces central venous pressure. In IVC occlusion therapy, the section of IVC inferior to the renal veins is temporarily occluded to, again, restrict blood volume entering the central venous system, which in turn reduces central venous pressure. A reduction in central venous pressure generates an increased pressure gradient across the kidneys, increasing kidney perfusion and/or increasing efficacy of diuretic therapies while simultaneously unloading the pressure on the heart to help restore normal cardiac function.

Some examples presented herein relate to prosthetic valve systems for reducing blood flow through a blood vessel (e.g., the SVC and/or IVC). In some examples, a prosthetic valve for placement in a blood vessel can comprise multiple leaflets configured to selectively open and close to reduce and/or not fully prevent blood flow through the prosthetic valve. Some valve leaflets may have one or more positions between being fully closed and fully open. In some examples, a system may comprise one or more sensors and/or controllers. A sensor coupled to a prosthetic valve may be configured to monitor pressure within a chamber (e.g., the right atrium). In some examples, a controller may be configured to provide power to the prosthetic valve and/or selectively control leaflets of the valve in response to pressure readings from the one or more sensors. The controller may be configured to move the leaflets between multiple predefined configurations. In some examples, the controller may be configured to individually control the leaflets.

In some examples, a prosthetic valve may comprise latching mechanisms to hold and/or lock the leaflets in place after being moved by the controller. The latching mechanisms may be reversible to allow the controller to overcome the latching mechanisms. In some examples, the controller may be configured to turn off power to the prosthetic valve when not actively moving the leaflets.

Some examples presented herein relate to venous occlusion therapy using fully implantable and/or electronically controlled flow restrictors for the treatment of acute heart failure. In some examples, therapy may involve occlusion of the SVC.

Some methods of SVC occlusion therapy can involve using one or more balloons to occlude the SVC. In some cases, open access points may be required for a device to hook up to a balloon pump and/or pressure monitor. Moreover, some devices may be non-implantable, which may require hospital readmissions. Some methods of IVC occlusion therapy can involve IVC occlusion catheters. However, such methods may require regular hospital readmissions and/or can provide poor long-term decongestive results.

Some examples presented herein advantageously can eliminate excessive hospital readmissions and/or can provide for a long-term decongestive therapy, improving both quality of life and overall survival rates, and/or with a lower cost to the healthcare system.

Example fully implantable and/or flow restrictive devices are presented herein for blood flow occlusion therapy. In some examples, devices may be delivered to the SVC via a subclavian and/or transfemoral approach. Additionally or alternatively, one or more devices may be delivered to the IVC (e.g., inferior to the renal veins) via a transfemoral approach. However, example devices may be delivered to any anatomical location clinically determined to provide effective placement.

In some examples, one or more devices may be implanted in a normally open position and/or may be shape set to an open position. For example, flow restriction may occur when the device receives a signal (e.g., current) from a separate and/or remote electronic controller. Such devices can provide relatively safe operation. For example, in the case of a device malfunction or failure, the device may not impede the blood flow pathways.

Some example devices may involve use of timing and/or activation signals controlled via an implantable microcontroller. For example, an Arduino Uno may be used. To power the microcontroller and flow restrictive device, a small and/or inductive wireless-rechargeable battery may be implanted. Additionally or alternatively, power by proxy devices may be used to allow for a greater physical distance between the patient and charging station. In some examples, an implantable battery may be omitted and replaced with an inductive and/or magnetic resonance receiver coil to supply power to the implant only when activated via an external power source. An implantable and/or rechargeable battery may be implanted and/or charged via magnetic resonance from a source inside and/or outside the body. This may allow for greater safety to the patient as during a possible failure mode the power source (a power source external to the patient's body) can be easily mechanically removed, forcing the device to shut off rather than software controlling the device to shutoff.

In some examples, implantable pressure sensors may be integrated into one or more implant and/or controllers. Pressure sensors may be configured to send data transmission via any suitable method, including end-to-end encryption cloud services and/or encrypted low-power Bluetooth technology. In some examples, data may be interrogated manually without cloud and/or wireless connectivity at an implanted device.

Patients in heart failure with preserved ejection fraction (HFpEF) exhibit exercise intolerance and a significant inverse relationship between exercise pulmonary capillary wedge pressure (PCWP) and peak oxygen uptake (VO2). Something as simple as a passive leg raise results in significant increase in PCWP. Conversely, studies of patients in heart failure with reduced ejection fraction (HFrEF) have failed to identify any correlation between cardiac filling pressures and peak VO2. Furthermore, looking at the volume overload profiles of patients with HFpEF and HFrEF, HFpEF patients hold their extra volume in the interstitial space, while HFrEF patients hold their extra volume in the vasculature. Taking these learnings into consideration, we have identified that not all congestion is the same. A possible complementary mechanism to the traditional congestion, secondary to sodium and fluid retention, is at play for patients with HFpEF. This possible mechanism is the mobilization of the venous reservoir resulting in rapid fluid shifts and subsequent hemodynamic measurements. HFpEF and/or HFrEF patients may benefit from a therapy that maintains kidney venous decongestion.

Expanding upon the venous occlusion therapy previously described, some example systems presented herein relate to a flow restrictor placed in the major splanchnic veins (e.g., hepatic veins) and/or used to regulate blood flow from the splanchnic reservoir. The flow restrictor may be used in conjunction with hemodynamic monitors to actively or passively adjust the amount of flow in response to cardiac filling pressures. For example, when a HFpEF patient undergoes a passive leg raise and subsequent PCWP rises, the flow restrictor may constrict and/or reduce the amount of blood mobilizing from the splanchnic reservoir, reducing cardiac filling pressures and PCWP. Additionally, the flow restrictor may be placed laparoscopically as a ring that wraps around the outside of the vein.

Some heart valve replacements (both surgical and transcatheter) involve use of passive leaflet motion. Such passive leaflet motion can operate at two discrete functions: fully open to allow blood flow, or fully closed to retard blood flow.

Example systems described herein can advantageously provide electronic actuators that can control leaflet timing and duration. Such systems can allow for finely tuning valve function to improved performance and efficiency.

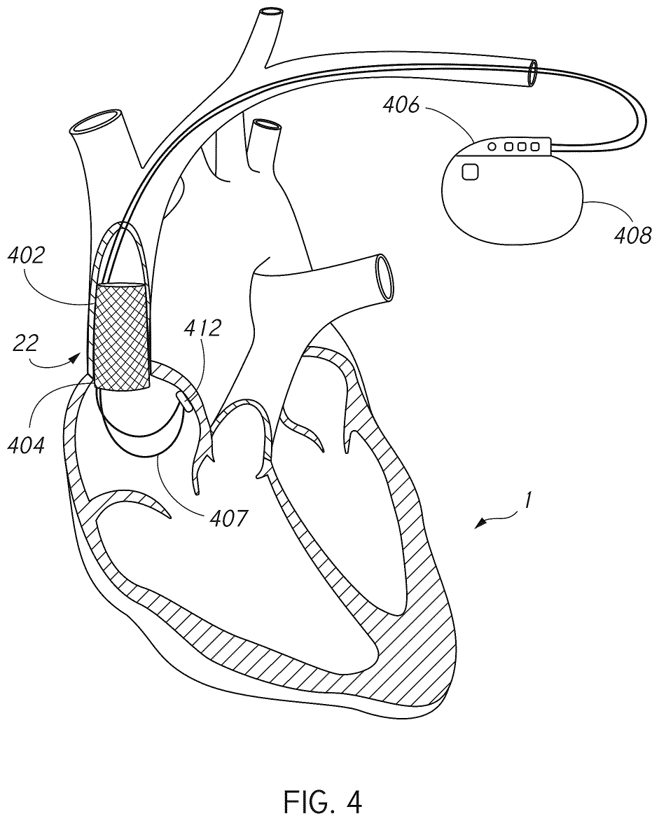

FIG. 4 illustrates an example system for modulating blood flow through one or more blood vessels, which can include the IVC and/or SVC 22 of a heart 1. In some examples, the system comprises one or more valves 402 and/or prosthetic valve systems configured for delivery and/or anchoring at least partially within the IVC 10, SVC 22, and/or other blood vessels. The valve 402 may comprise any implant (e.g., a stent) configured for placement within a blood vessel and/or configured for at least partially occluding blood flow through the blood vessel. While the valve 402 is shown disposed within the SVC 22, one or more valves 402 may additionally or alternatively be delivered to other anatomical locations. A valve 402 can comprise one or more components, which can include a frame 404 and/or one or more leaflets disposed within a lumen formed by the frame 404. The valve system can further include one or more controllers 406, batteries 408, and/or blood pressure monitoring sensors 412. In some examples, one or more components may be combined. For example, a battery 408 and a controller 406 may have a unitary structure. In some examples, some components of the system may be disposed outside of a patient's body. For example, the valve 402 and/or sensor(s) may be disposed within a patient's body and/or heart and/or the controller 406 and/or battery 408 may be disposed outside the patient's body. While a valve 402 is shown within the SVC 22, one or more valves 402 may be placed in the IVC (e.g., at or near one or more hepatic veins) and/or below the kidneys.

In some examples, the valve 402 may be at least partially dynamic and/or adjustable. For example, one or more leaflets of the valve 402 may be configured to be adjusted and/or locked in place in different positions. In some examples, the dynamic valve 402 may not completely close off the SVC 22 and/or leaflets of the valve 402 may be adjusted via the controller 406 in response to pressure changes detected by the sensor 412 so that the flow of blood to the right atrium 5 is reduced.

Reduction of blood flow and/or blood pressure may be accomplished in any suitable way. In some examples, the valve 402 may comprise one or more leaflets configured to be adjusted to and/or between multiple (e.g., two or three) different and/or predefined positions. The leaflets may be moved and/or adjusted between the multiple positions in response to blood flow and/or pressure changes detected by the sensor 412. For example, when blood pressure exceeds a threshold level, the valve 402 may adjust (e.g., leaflets of the valve 402 may adjust) to increase an amount of blood flow occlusion of the valve 402. The valve 402 may continue to adjust further in response to blood pressure levels maintaining an elevated level and/or increasing beyond a second and/or additional threshold level.

In some examples, the valve 402 may comprise three or more leaflets. The multiple leaflets may be individually and/or jointly actuated and/or activated in response to increased blood flow. An amount of adjustment may depend on an amount of needed blood flow reduction.

Actively maintaining a position of the one or more leaflets following adjustment by the controller 406 may require significant power from the battery 408 without features to allow the controller to 406 shut off following an adjustment. Leaflet adjustments may be required to be maintained for up to twelve hours at a time, creating an excessive strain on the controller 406 and/or battery 408.

To improve effectiveness and/or efficiency of the controller 406 and/or battery 408, one or more leaflets of the valve 402 may be held and/or locked in place using one or more latching mechanisms and/or means for latching. A latching mechanism and/or means for latching can include any latches, pegs, knobs, notches, protrusions, arms, fingers, cavities, discs, and/or similar mechanisms configured to prevent and/or resist movement and/or adjustment of the one or more leaflets beyond a given position. In some examples, one or more latching mechanisms may be at least partially reversible and/or may be configured to recede in response to increased force from the leaflets.

In some examples, one or more leaflets may be moved into a desired position (e.g., causing a first amount of blood flow reduction) and/or an actuatable (e.g., spinnable) portion of the valve may be actuated to latch the one or more leaflets at the desired position. In another example, one or more hard stop latches (e.g., cavities and/or protrusions) may be placed along the valve 402 (e.g., along an inner and/or outer surface of the frame 404) to receive the one or more leaflets at predetermined positions. The one or more leaflets can be configured to engage a reversible locking mechanism (e.g., a détente and/or other latching mechanism) configured to hold the leaflet in place while allowing the leaflets to be repositioned by overcoming the reversible locking mechanism. Once the leaflet(s) are in place, power to the leaflet(s) may be turned off to preserve battery life.

The system may be controlled in a closed loop circuit. For example, a closed-loop feedback system may be used to control the valve 402. The system may initially be in a sensing mode, in which pressure sensor(s) 412 inferior and/or superior to the valve 402 (e.g., located in the right atrium 5, SVC 22, and/or IVC) are used to monitor pressures and/or gradients across the valve 402 and/or send pressure date to the controller 406. The controller 406 may then calculate optimal filling pressures and/or required current to open, close, and/or partially close the valve 402. The system may be configured to switch into activation mode upon completion of the sensing mode. In activation mode, the controller 406 may be configured to adjust the valve 402 to the previously calculated values to achieve optimal filling pressures. The system may be configured to switch back to the sensing mode to begin the closed-loop cycle again. Additionally, the controller 406 may have its power supplied via wireless induction or an implantable battery 408. The controller 406 may also have a peripheral device outside the patient used to transmit data to and/or from the controller 406. The transmitted data may be used to monitor the system and/or its critical care sensors 412. This data then can be uploaded to the patient's health care provider to allow for long term data acquisition and/or device management.

In some examples, the sensor 412 may be coupled to the valve 402 and/or controller 406 via one or more actuation wires 407. While two actuation wires 407 are shown in FIG. 4, the system may comprise any number of actuation wires 407. Actuation wires 407 may be configured to conduct electronic signals (e.g., currents) from the sensor 412 to the controller 406 and/or valve 402 and/or from the controller 406 to the valve 402 and/or sensor 412. In some examples, the controller 406 may be configured to transmit one or more signals to the valve 402 based on and/or in response to signals received at the controller 406 and/or from the sensor 412.

The controller 406 and/or other device may be configured to transmit one or more control signals to the valve 402 to cause and/or control movement of the valve 402. For example, a control signal may cause one or more actuation wires 407 to move, which may cause corresponding movement of one or more leaflets and/or other components of the valve 402. In some examples, transmission of one or more control signals may be based at least in part on one or more blood flow signals received (e.g., at the controller 406) from the sensor 412. For example, the sensor 412 may be configured to transmit blood flow signals related to blood flow and/or pressure within the heart 1 via the actuation wires 407 to the controller 406 and/or other device. The actuation wires 407 may be configured to receive and/or transmit control and/or flow signals to and/or from the controller 406, valve 402, and/or sensor 412.

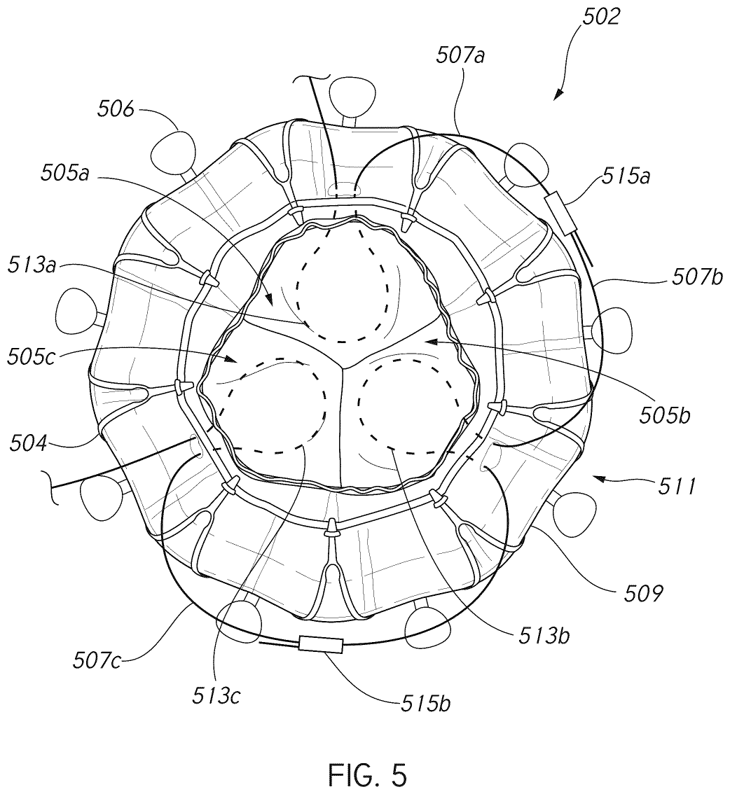

FIG. 5 illustrates an example occlusion valve 502 configured to dynamically occlude one or more blood vessels of a heart in accordance with one or more examples. The valve 502 is shown in a closed form. The valve 502 may comprise one or more leaflets 505 configured to extend at least partially across a lumen of the valve 502. For example, the valve 502 and/or a frame 504 of the valve 502 may have a generally cylindrical form extending around a generally cylindrical inner lumen. The inner lumen may be configured to allow blood flow through the valve 502. The one or more leaflets 505 may be configured to selectively open and/or close the inner lumen of the valve 502. The valve 502 is shown in FIG. 5 in a fully closed form in which the one or more leaflets 505 extend fully across the lumen of the valve 502. However, the one or more leaflets 505 may be configured to dynamically adjust in position to at least partially open the lumen and/or to create an opening through the leaflets 505. The valve may comprise a skirt 509 configured to extend at least partially along the frame 504 of the valve 502. The frame 504 may comprise a wire stent comprising a network of struts forming one or more cells. In some examples, the skirt 509 may be configured to extend across one or more cells and/or struts formed by the frame 504. The skirt 509 may be at least partially composed of tissue, cloth, fabric, and/or any suitable material and/or may have a generally flexible structure.

The frame 504 of the valve 502 may form one or more flanges 511. For example, the valve 502 may comprise a flange 511 at a distal end of the valve 502. The valve 502 may additionally or alternatively comprise a flange 511 at a proximal end of the valve 502. The flange 511 may comprise a flared and/or jutted out portion of the frame 504 configured to increase a diameter of the valve 502 at the distal and/or proximal ends of the valve 502. For example, the flange 511 may be configured to extend at least partially into a right atrium and/or other chamber and/or blood vessel of a heart. In some examples, the skirt 509 may be configured to extend at least partially along the flange 511.

In the example shown in FIG. 5, the valve 502 comprises three leaflets. However, example valves 502 may comprise other numbers of leaflets 505 (e.g., two or four). The one or more leaflets 505 may extend in series around a circumference of the valve 502 and/or frame 504. For example, each of the leaflets 505 may extend from a different portion of a circumference of the valve 502 and/or frame 504. In some examples, the one or more leaflets 505 may at least partially overlap and/or contact each other in a fully closed and/or partially closed form of the valve 502.

In some examples, positions of the leaflets 505 may be dynamically adjusted using various components of a modulation system and/or various components of the valve 502. For example, a controller may transmit electronic signals and/or pulses from a battery to the leaflets 505 via one or more actuation wires 507. The actuation wires 507 may be composed of any suitable material(s), including Nitinol and/or other shape memory alloys. In some examples, the actuation wires 507 may be at least partially conductive and/or may comprise one or more conductive wires configured to conduct electric signals and/or pulses from one or more controllers and/or batteries to the actuation wires 507. An actuation wire 507 can comprise any wire, cord, arm, elongate member, and/or similar device configured to conduct electric signals and/or support one or more leaflets 505.

The actuation wires 507 may be shape set in a desired form. For example, the one or more actuation wires 507 may be configured to loop through one or more portions of the skirt 509 and/or frame 504. In some examples, the actuation wires 507 may be configured to extend along and/or under one or more leaflets 505 of the valve 502 to support the leaflets 505. The actuation wires 507 may be shape set into a loop 513 (e.g., a horseshoe loop) and/or similar shape along an underside of one or more leaflets 505.

In some examples, multiple actuation wires 507 may be configured to support the leaflets 505 of the valve 502. For example, a first actuation wire 507a may pass through the skirt 509 at or near a first leaflet 505a and/or form a first loop 513a under the first leaflet 505a. The first actuation wire 507a may exit through the skirt 509 at or near the first leaflet 505a. The first actuation wire 507a may be joined to a second actuation wire 507b via first coupler 515a. The second actuation wire 507b may pass through the skirt 509 at or near a second leaflet 505b and/or form a second loop 513b under the second leaflet 505b. The second actuation wire 507b may exit through the skirt 509 at or near the second leaflet 505b. The second actuation wire 507b may be joined to a third actuation wire 507c via second coupler 515b. The third actuation wire 507c may pass through the skirt 509 at or near a third leaflet 505c and/or form a third loop 513c under the third leaflet 505c. The third actuation wire 507c may exit through the skirt 509 at or near the third leaflet 505c.

Alternatively, a single actuation wire 507 may be configured to support each of the leaflets 505 of the valve 502. For example, the couplers 515 may not be used and instead the single actuation wire 507 may extend into and out of the skirt 509 as shown in FIG. 5 to extend along each of the leaflets 505.

The one or more actuation wires 507 may be configured to form a scaffold that closes the leaflet(s) 505 when current is applied to the actuation wires 507 and/or other conductive wires coupled to the actuation wires 507. For example, the actuation wires 507 may be configured to extend upwardly and/or towards the flange 511 in response to a current, thus closing the inner lumen of the valve 502. To open the valve 502, pressure built up from occluding blood flow through a blood vessel may overcome a resistance of the leaflets 505 and/or actuation wires 507, causing the leaflets 505 and/or actuation wires 507 to recede and/or form an opening through the valve 502. Additionally or alternatively, one or more devices and/or mechanisms may be configured to force open the leaflets 505. For example, an elastic (e.g., super elastic) wire (e.g., composed at least partially of Nitinol and/or other shape memory alloys) can act as a spring to force open the leaflets 505.

In some examples, the valve 502 may be configured for delivery and/or use at the SVC and/or IVC of a heart. For example, the valve 502 may be disposed inferior to one or more renal veins. The valve 502 may be configured to provide intermittent occlusion of one or more blood vessels, including the SVC and/or IVC.

The one or more actuation wires 507 may be configured to be powered via one or more actuators and/or controllers. The one or more actuation wires 507 may be attached to the one or more leaflets 505 and/or may be coupled to and/or affixed to the frame 504 of the valve 502. In some examples, one or more actuation wires 507 and/or actuators may be disposed within silicone tubes. One or more actuators and/or controllers can be affixed and/or coupled to one or more mounts of the frame 504.

In some examples, the one or more leaflets 505 may be configured to move independently. For example, individual actuation wires 507 may be separately attached to separate leaflets 505 and/or may be configured to move in response to different signals and/or pulses from an actuator and/or controller. Additionally or alternatively, a single actuation wire 507 may be configured to control different leaflets 505 differently and/or independently. In some examples, one or more leaflets 505 may be configured to be moved together (e.g., in a synchronized manner) and/or approximately simultaneously.

The valve 502 may be configured to selectively modulate blood flow through one or more blood vessels in which the valve 502 may be disposed. In some examples, the valve 502 may be configured to be fully implanted within a blood vessel and/or may not be tethered to a catheter and/or other delivery devices.

In some examples, the valve 502 may be biased towards an open state. For example, the leaflets 505 and/or actuation wires 507 may be biased to extend generally in parallel with the frame 504. In response to signals and/or pulses from one or more actuators and/or controllers, the leaflets 505 and/or actuation wires 507 may be configured to extend into the generally perpendicular and/or closed form with respect to the frame 504 shown in FIG. 5. The one or more actuation wires 507 may be configured to form paddles extending at least partially along the one or more leaflets 505.

One or more actuation wires 507 may be configured to responsive to changes in temperature. For example, the one or more actuation wires 507 may be configured to extend into the closed form in response to temperature at the valve 502 exceeding a threshold temperature value. The actuation wires 507 may be shape set to be responsive to a predetermined threshold temperature. The actuation wires 507 may be set to maintain a closed and/or relaxed position in response to temperatures at or moderately above normal body temperature. In some examples, the one or more actuation wires 507 may be heated with a current to increase the temperature of the actuation wires 507 and/or to cause the one or more actuation wires 507 to extend into the closed form.

In some examples, the one or more actuation wires 507 may be configured to be controlled and/or powered wirelessly via a cloud server at an external location and/or situated remotely from the patient's body. A wireless charger may be used to wirelessly supply power to one or more actuators and/or controllers within the patient's body. Current may be continuously supplied to the one or more actuation wires 507 to maintain a closed state of the leaflets 505.

The valve 502 may comprise various features and/or mechanisms configured to reopen the valve 502 after the leaflets 505 move to an at least partially closed form. In one example, the valve 502 may comprise mechanical pull wires (e.g., fingers) configured to contract and/or pull the leaflets 505 back into an open state. In another example, one or more springs (e.g., composed at least partially of Nitinol and/or other shape memory alloys) may be configured to collapse when heated to pull the leaflets 505 back.

In some examples, the valve 502 may comprise one or more latching mechanisms configured to hold the leaflets 505 in one or more at least partially closed forms to reduce an amount of power used by the valve 502. In one example, the valve 502 may comprise one or more pins and/or rotation mechanisms configured to lock the leaflets 505 in a particular position. In another example, one or more magnetic elements may be configured to mate with corresponding magnetic elements attached to the leaflets 505 and/or may be positioned at different locations along the valve 502 to hold the leaflets 505 in particular positions. The valve 502 may comprise multiple types of latching mechanisms, including active and/or passive latching mechanisms. The valve 502 may comprise one or more latching mechanisms configured to quickly release the leaflets 505 in response to signals. For example, in emergency situations, the one or more latching mechanisms may be configured to recede and/or release the leaflets 505.

The valve 502 may comprise one or more arms 506 configured for anchoring and/or placing the valve 502 at a target location. The one or more arms 506 may have generally curved forms and/or may be configured to extend at least partially above the flange 511 of the valve 502.

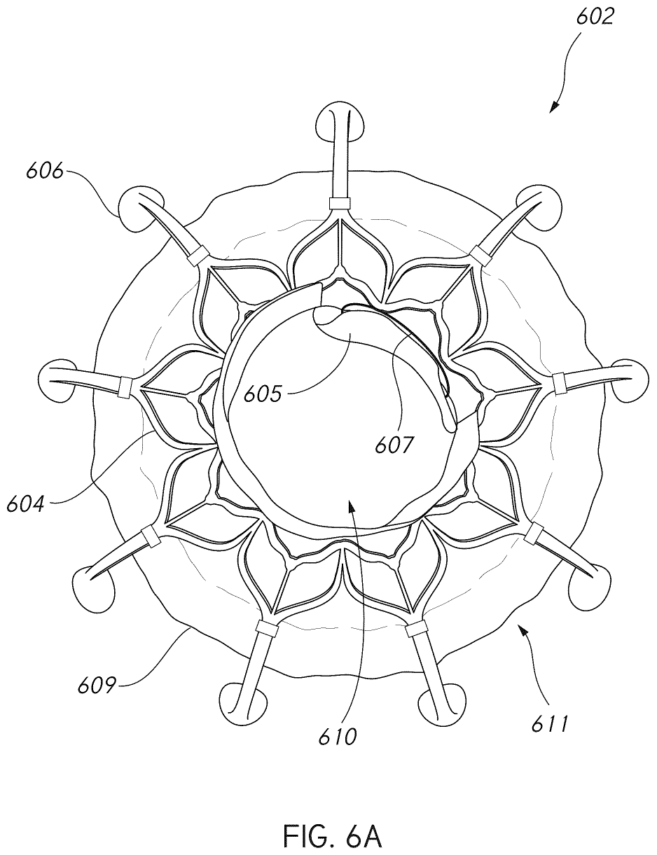

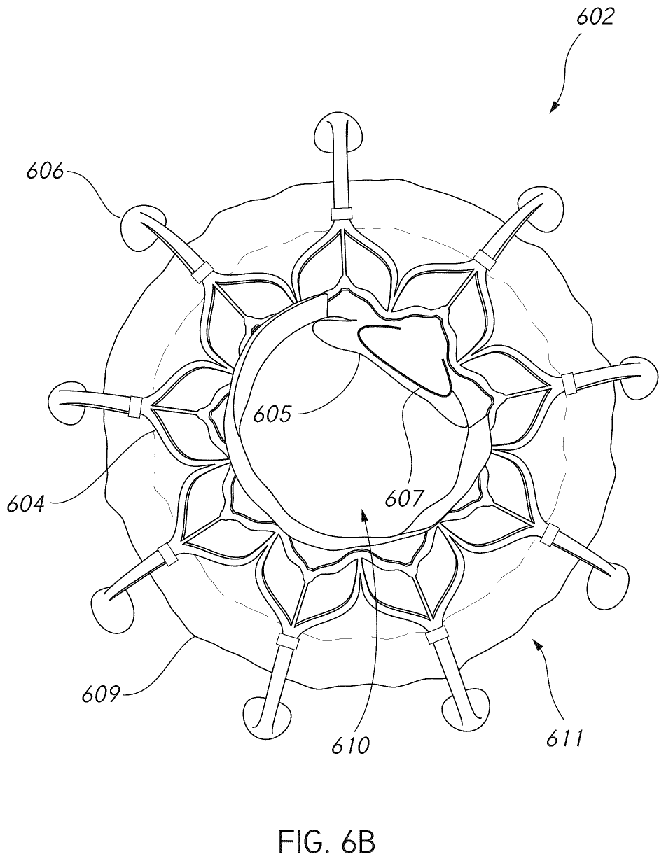

FIG. 6A-6C provide overhead views of an example occlusion valve 602 configured to dynamically occlude one or more blood vessels of a heart in accordance with one or more examples. The valve 602 may comprise one or more leaflets 605, one or more actuation wires 607, a frame 604, and/or a skirt 609. In FIG. 6A, the valve 602 is shown in an open state, in which the one or more leaflets 605 extend generally in parallel with cylindrical sides of the frame 604 and/or generally away from an inner lumen 610 of the valve 602. In FIG. 6B, the valve 602 is shown in a first partially closed form, in which the one or more leaflets 605 extend at least partially over the lumen 610, at an angle with the cylindrical sides of the frame 604, and/or towards each other. In FIG. 6C, the valve 602 is shown in a second partially closed form, in which the one or more leaflets 605 extend further over the lumen 610, at greater angles with the cylindrical sides of the frame 604, and/or closer towards each other.

Movement of the leaflets 605 from the open form to the partially closed and/or closed forms may be caused at least in part by the one or more actuation wires 607. The one or more actuation wires 607 may be configured to extend at least partially along the one or more leaflets 605. In some examples, the one or more actuation wires 607 may be attached, affixed, and/or coupled to the one or more leaflets 605. For example, the one or more leaflets 605 may comprise flaps 616 and/or folds configured to at least partially enclose the actuation wires 607 and/or secure the actuation wires 607 to the leaflets 605.

The valve 602 may comprise a flange 611 at distal and/or proximal end of the valve 602. The flange 611 may have an increased diameter relative to other portions of the valve 602.

In some examples, the valve 602 may comprise one or more arms 606, which may be configured to facilitate anchoring of the valve 602 to a blood vessel and/or chamber of a heart. The one or more arms 606 may have generally curved forms. For example, the one or more arms 606 may be configured to extend from a distal end of the valve 602 towards the proximal end of the valve 602 along an outer surface of the valve 602.

The one or more actuation wires 607 may be disposed along any suitable surface of the one or more leaflets 605 and/or may be configured to extend at least partially along and/or through the frame 604 and/or skirt 609. For example, as shown in FIGS. 6A-6C, the one or more actuation wires 607 may be configured to extend along an upper side of the leaflets 605 and/or may be configured to pull the leaflets 605 upwardly towards the distal end of the valve 602. Additionally or alternatively, one or more actuation wires 607 may be configured to extend along an underside of the one or more leaflets 605 and/or may be configured to press the one or more leaflets 605 upwardly towards the distal end of the valve 602.

The leaflets 605 of the valve 602 may be held in the first partially closed form and/or second partially closed form through use of one or more latching mechanisms. For example, the frame 604 and/or skirt 609 may comprise one or more pegs, notches, protrusions, recesses, discs, springs, and/or similar mechanisms configured to grasp, hold, and/or receive the one or more leaflets 605 in desired positions.

The one or more actuation wires 607 may be configured to form a scaffold that closes the leaflet(s) 605 when current is applied to the actuation wires 607 and/or other conductive wires coupled to the actuation wires 607. For example, the actuation wires 607 may be configured to extend upwardly and/or towards the flange 611 in response to a current, thus closing the inner lumen of the valve 602. To open the valve 602, pressure built up from occluding blood flow through a blood vessel may overcome a resistance of the leaflets 605 and/or actuation wires 607, causing the leaflets 605 and/or actuation wires 607 to recede and/or form an opening through the valve 602. Additionally or alternatively, one or more devices and/or mechanisms may be configured to force open the leaflets 605. For example, an elastic (e.g., super elastic) wire (e.g., composed at least partially of Nitinol and/or other shape memory alloys) can act as a spring to force open the leaflets 605.

In some examples, the valve 602 may be configured for delivery and/or use at the SVC and/or IVC of a heart. For example, the valve 602 may be disposed inferior to one or more renal veins. The valve 602 may be configured to provide intermittent occlusion of one or more blood vessels, including the SVC and/or IVC.

The one or more actuation wires 607 may be configured to be powered via one or more actuators and/or controllers. The one or more actuation wires 607 may be attached to the one or more leaflets 605 and/or may be coupled to and/or affixed to the frame 604 of the valve 602. In some examples, one or more actuation wires 607 and/or actuators may be disposed within silicone tubes. One or more actuators and/or controllers can be affixed and/or coupled to one or more mounts of the frame 604.

In some examples, the one or more leaflets 605 may be configured to move independently. For example, individual actuation wires 607 may be separately attached to separate leaflets 605 and/or may be configured to move in response to different signals and/or pulses from an actuator and/or controller. Additionally or alternatively, a single actuation wire 607 may be configured to control different leaflets 605 differently and/or independently. In some examples, one or more leaflets 605 may be configured to be moved together and/or approximately simultaneously.

The valve 602 may be configured to selectively modulate blood flow through one or more blood vessels in which the valve 602 may be disposed. In some examples, the valve 602 may be configured to be fully implanted within a blood vessel and/or may not be tethered to a catheter and/or other delivery devices.

In some examples, the valve 602 may be biased towards an open state. For example, the leaflets 605 and/or actuation wires 607 may be biased to extend generally in parallel with the frame 604. In response to signals and/or pulses from one or more actuators and/or controllers, the leaflets 605 and/or actuation wires 607 may be configured to extend into the generally perpendicular and/or closed form with respect to the frame 604 shown in FIG. 6. The one or more actuation wires 607 may be configured to form paddles extending at least partially along the one or more leaflets 605.

One or more actuation wires 607 may be configured to responsive to changes in temperature. For example, the one or more actuation wires 607 may be configured to extend into the closed form in response to temperature at the valve 602 exceeding a threshold temperature value. The actuation wires 607 may be shape set to be responsive to a predetermined threshold temperature. The actuation wires 607 may be set to maintain a closed and/or relaxed position in response to temperatures at or moderately above normal body temperature. In some examples, the one or more actuation wires 607 may be heated with a current to increase the temperature of the actuation wires 607 and/or to cause the one or more actuation wires 607 to extend into the closed form.

In some examples, the one or more actuation wires 607 may be configured to be controlled and/or powered wirelessly via a cloud server at an external location and/or situated remotely from the patient's body. A wireless charger may be used to wirelessly supply power to one or more actuators and/or controllers within the patient's body. Current may be continuously supplied to the one or more actuation wires 607 to maintain a closed state of the leaflets 605.

The valve 602 may comprise various features and/or mechanisms configured to reopen the valve 602 after the leaflets 605 to an at least partially closed form. In one example, the valve 602 may comprise mechanical pull wires (e.g., fingers) configured to contract and/or pull the leaflets 605 back into an open state. In another example, one or more springs (e.g., composed at least partially of Nitinol and/or other shape memory alloys) may be configured to collapse when heated to pull the leaflets 605 back.

In some examples, the valve 602 may comprise one or more latching mechanisms configured to hold the leaflets 605 in one or more at least partially closed forms to reduce an amount of power used by the valve 602. The latching mechanisms may be configured to hold the leaflets 605 in various predetermined positions, including each of the positions illustrated in FIGS. 6A-6C. In one example, the valve 602 may comprise one or more pins and/or rotation mechanisms configured to lock the leaflets 605 in a particular position. In another example, one or more magnetic elements may be configured to mate with corresponding magnetic elements attached to the leaflets 605 and/or may be positioned at different locations along the valve 602 to hold the leaflets 605 in particular positions. The valve 602 may comprise multiple types of latching mechanisms, including active and/or passive latching mechanisms. The valve 602 may comprise one or more latching mechanisms configured to quickly release the leaflets 605 in response to signals. For example, in emergency situations, the one or more latching mechanisms may be configured to recede and/or release the leaflets 605.

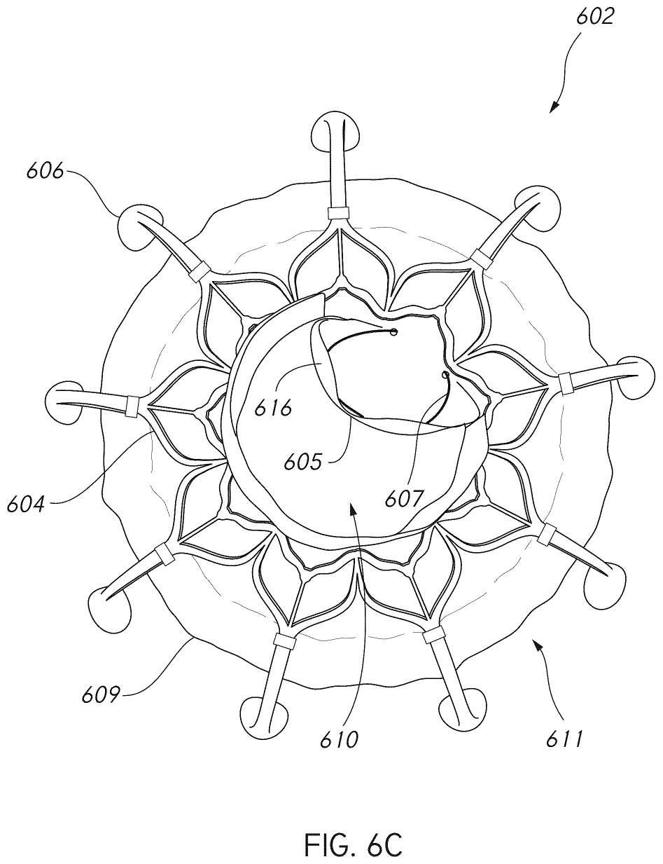

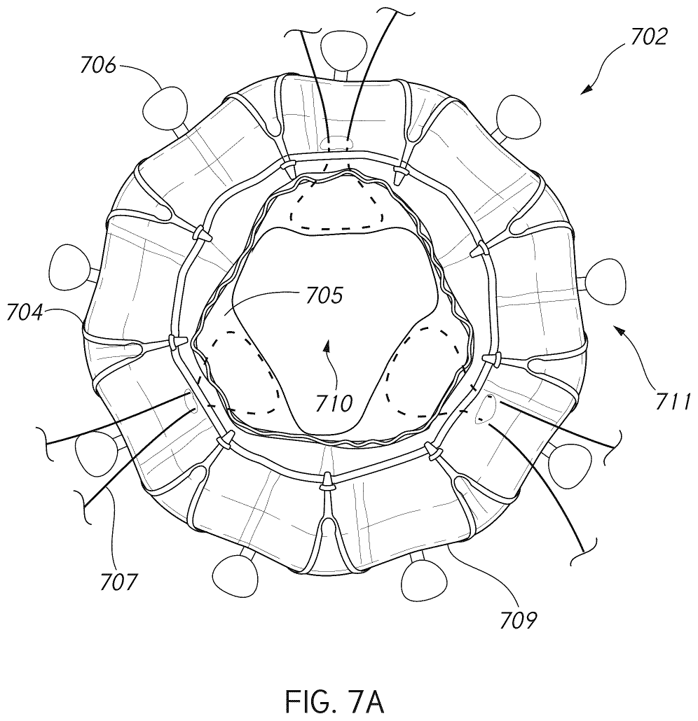

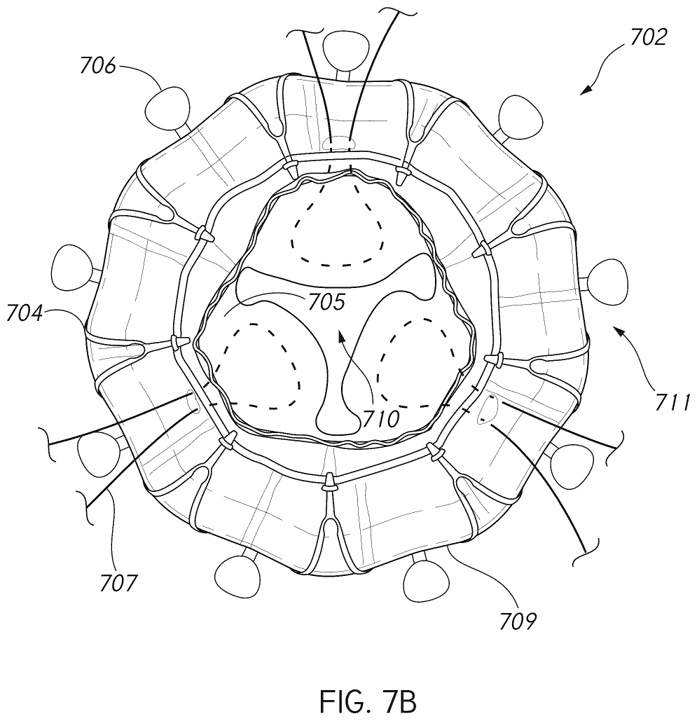

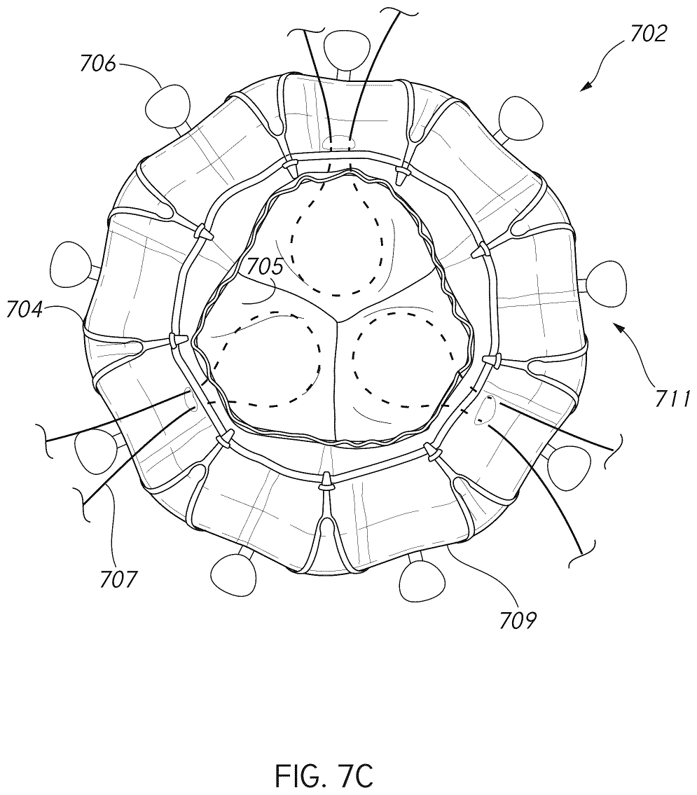

FIGS. 7A-7C provide overhead views of an example occlusion valve 702 configured to dynamically occlude one or more blood vessels of a heart in accordance with one or more examples. The valve 702 may comprise one or more leaflets 705, one or more actuation wires 707, a frame 704, and/or a skirt 709. In FIG. 7A, the valve 702 is shown in an open state, in which the one or more leaflets 705 extend generally in parallel with cylindrical sides of the frame 704 and/or generally away from an inner lumen 710 of the valve 702. In FIG. 7B, the valve 702 is shown in a partially closed form, in which the one or more leaflets 705 extend at least partially over the lumen 710, at an angle with the cylindrical sides of the frame 704, and/or towards each other. In FIG. 7C, the valve 702 is shown in a fully closed form, in which the one or more leaflets 705 extend fully across the lumen 710 and/or fully cover the lumen 710.

The one or more actuation wires 707 may be configured to form a scaffold that closes the leaflet(s) 705 when current is applied to the actuation wires 707 and/or other conductive wires coupled to the actuation wires 707. For example, the actuation wires 707 may be configured to extend upwardly and/or towards the flange 711 in response to a current, thus closing the inner lumen of the valve 702. To open the valve 702, pressure built up from occluding blood flow through a blood vessel may overcome a resistance of the leaflets 705 and/or actuation wires 707, causing the leaflets 705 and/or actuation wires 707 to recede and/or form an opening through the valve 702. Additionally or alternatively, one or more devices and/or mechanisms may be configured to force open the leaflets 705. For example, an elastic (e.g., super elastic) wire (e.g., composed at least partially of Nitinol and/or other shape memory alloys) can act as a spring to force open the leaflets 705.

In some examples, the valve 702 may be configured for delivery and/or use at the SVC and/or IVC of a heart. For example, the valve 702 may be disposed inferior to one or more renal veins. The valve 702 may be configured to provide intermittent occlusion of one or more blood vessels, including the SVC and/or IVC. The valve 702 may comprise one or more anchoring arms 706 configured to facilitate anchoring of the valve 702 at the SVC, IVC, and/or other location.

The one or more actuation wires 707 may be configured to be powered via one or more actuators and/or controllers. The one or more actuation wires 707 may be attached to the one or more leaflets 705 and/or may be coupled to and/or affixed to the frame 704 of the valve 702. In some examples, one or more actuation wires 707 and/or actuators may be disposed within silicone tubes. One or more actuators and/or controllers can be affixed and/or coupled to one or more mounts of the frame 704.

In some examples, the one or more leaflets 705 may be configured to move independently. For example, individual actuation wires 707 may be separately attached to separate leaflets 705 and/or may be configured to move in response to different signals and/or pulses from an actuator and/or controller. Additionally or alternatively, a single actuation wire 707 may be configured to control different leaflets 705 differently and/or independently. In some examples, one or more leaflets 705 may be configured to be moved together and/or approximately simultaneously.

The valve 702 may be configured to selectively modulate blood flow through one or more blood vessels in which the valve 702 may be disposed. In some examples, the valve 702 may be configured to be fully implanted within a blood vessel and/or may not be tethered to a catheter and/or other delivery devices.

In some examples, the valve 702 may be biased towards an open state. For example, the leaflets 705 and/or actuation wires 707 may be biased to extend generally in parallel with the frame 704. In response to signals and/or pulses from one or more actuators and/or controllers, the leaflets 705 and/or actuation wires 707 may be configured to extend into the generally perpendicular and/or closed form with respect to the frame 704 shown in FIG. 7. The one or more actuation wires 707 may be configured to form paddles extending at least partially along the one or more leaflets 705.

One or more actuation wires 707 may be configured to responsive to changes in temperature. For example, the one or more actuation wires 707 may be configured to extend into the closed form in response to temperature at the valve 702 exceeding a threshold temperature value. The actuation wires 707 may be shape set to be responsive to a predetermined threshold temperature. The actuation wires 707 may be set to maintain a closed and/or relaxed position in response to temperatures at or moderately above normal body temperature. In some examples, the one or more actuation wires 707 may be heated with a current to increase the temperature of the actuation wires 707 and/or to cause the one or more actuation wires 707 to extend into the closed form.

In some examples, the one or more actuation wires 707 may be configured to be controlled and/or powered wirelessly via a cloud server at an external location and/or situated remotely from the patient's body. A wireless charger may be used to wirelessly supply power to one or more actuators and/or controllers within the patient's body. Current may be continuously supplied to the one or more actuation wires 707 to maintain a closed state of the leaflets 705.

The valve 702 may comprise various features and/or mechanisms configured to reopen the valve 702 after the leaflets 705 to an at least partially closed form. In one example, the valve 702 may comprise mechanical pull wires (e.g., fingers) configured to contract and/or pull the leaflets 705 back into an open state. In another example, one or more springs (e.g., composed at least partially of Nitinol and/or other shape memory alloys) may be configured to collapse when heated to pull the leaflets 705 back.

In some examples, the valve 702 may comprise one or more latching mechanisms configured to hold the leaflets 705 in one or more at least partially closed forms to reduce an amount of power used by the valve 702. In one example, the valve 702 may comprise one or more pins and/or rotation mechanisms configured to lock the leaflets 705 in a particular position. In another example, one or more magnetic elements may be configured to mate with corresponding magnetic elements attached to the leaflets 705 and/or may be positioned at different locations along the valve 702 to hold the leaflets 705 in particular positions. The valve 702 may comprise multiple types of latching mechanisms, including active and/or passive latching mechanisms. The valve 702 may comprise one or more latching mechanisms configured to quickly release the leaflets 705 in response to signals. For example, in emergency situations, the one or more latching mechanisms may be configured to recede and/or release the leaflets 705.

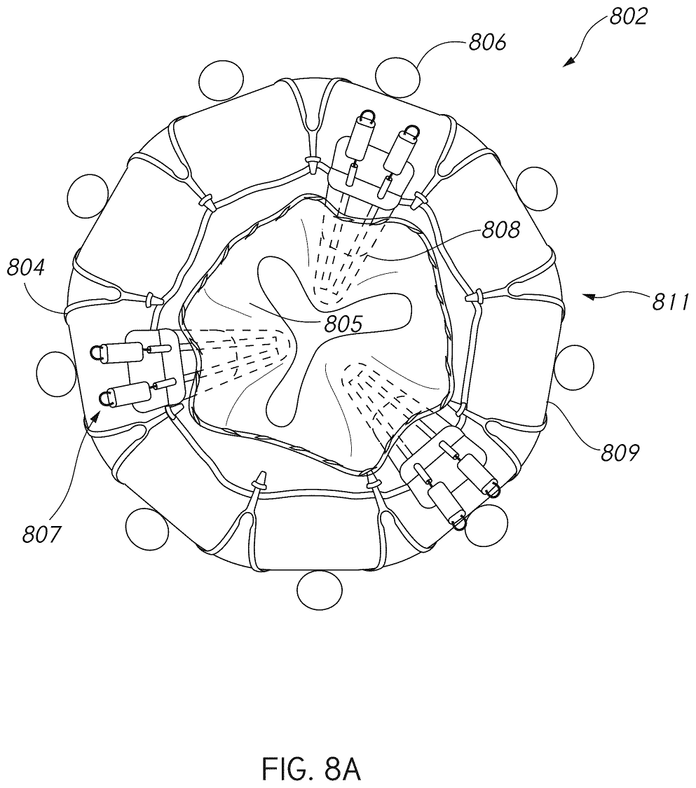

FIGS. 8A and 8B illustrate another example occlusion valve 802 configured to dynamically occlude one or more blood vessels of a heart in accordance with one or more examples. FIG. 8A provides an overhead view of the valve 802 and FIG. 8B provides bottom view of the valve 802. The valve 802 may comprise one or more leaflets 805, a frame 804, and/or a skirt 809. The valve 802 may further comprise one or more actuation members 808 (e.g., support arms), which can include wires, arms, elongate members, fingers, coils, and/or similar devices configured to support the one or more leaflets 805.

The one or more actuation members 808 may have a generally sturdy and/or rigid structure and/or may be configured to effectively support the one or more leaflets 805. In some examples, the one or more actuation members 808 may comprise one or more actuation wires and/or may be configured to couple to one or more actuation wires.

In some examples, the valve 802 may comprise one or more arms 806, which may be configured to facilitate anchoring of the valve 802 to a blood vessel and/or chamber of a heart. The one or more arms 806 may have generally curved forms. For example, the one or more arms 806 may be configured to extend from a distal end of the valve 802 towards the proximal end of the valve 802 along an outer surface of the valve 802.

The one or more actuation members 808 may be disposed along any suitable surface of the one or more leaflets 805 and/or may be configured to extend at least partially along and/or through the frame 804 and/or skirt 809. For example, the one or more actuation members 808 may be configured to extend along an upper side of the leaflets 805 and/or may be configured to pull the leaflets 805 upwardly towards the distal end of the valve 802. Additionally or alternatively, one or more actuation members 808 may be configured to extend along an underside of the one or more leaflets 805 and/or may be configured to press the one or more leaflets 805 upwardly towards the distal end of the valve 802.

The leaflets 805 of the valve 802 may be held in the first partially closed form and/or second partially closed form through use of one or more latching mechanisms. For example, the frame 804 and/or skirt 809 may comprise one or more pegs, notches, protrusions, recesses, discs, springs, and/or similar mechanisms configured to grasp, hold, and/or receive the one or more leaflets 805 in desired positions.

The one or more actuation wires 807 may be configured to form a scaffold that closes the leaflet(s) 805 when current is applied to the actuation wires 807 and/or other conductive wires coupled to the actuation wires 807. For example, the actuation wires 807 may be configured to extend upwardly and/or towards the flange 811 in response to a current, thus closing the inner lumen of the valve 802. To open the valve 802, pressure built up from occluding blood flow through a blood vessel may overcome a resistance of the leaflets 805 and/or actuation wires 807, causing the leaflets 805 and/or actuation wires 807 to recede and/or form an opening through the valve 802. Additionally or alternatively, one or more devices and/or mechanisms may be configured to force open the leaflets 805. For example, an elastic (e.g., super elastic) wire (e.g., composed at least partially of Nitinol and/or other shape memory alloys) can act as a spring to force open the leaflets 805.

In some examples, the valve 802 may be configured for delivery and/or use at the SVC and/or IVC of a heart. For example, the valve 802 may be disposed inferior to one or more renal veins. The valve 802 may be configured to provide intermittent occlusion of one or more blood vessels, including the SVC and/or IVC.

The one or more actuation wires 807 may be configured to be powered via one or more actuators and/or controllers. The one or more actuation wires 807 may be attached to the one or more leaflets 805 and/or may be coupled to and/or affixed to the frame 804 of the valve 802. In some examples, one or more actuation wires 807 and/or actuators may be disposed within silicone tubes. One or more actuators and/or controllers can be affixed and/or coupled to one or more mounts of the frame 804.

In some examples, the one or more leaflets 805 may be configured to move independently. For example, individual actuation wires 807 may be separately attached to separate leaflets 805 and/or may be configured to move in response to different signals and/or pulses from an actuator and/or controller. Additionally or alternatively, a single actuation wire 807 may be configured to control different leaflets 805 differently and/or independently. In some examples, one or more leaflets 805 may be configured to be moved together and/or approximately simultaneously.

The valve 802 may be configured to selectively modulate blood flow through one or more blood vessels in which the valve 802 may be disposed. In some examples, the valve 802 may be configured to be fully implanted within a blood vessel and/or may not be tethered to a catheter and/or other delivery devices.

In some examples, the valve 802 may be biased towards an open state. For example, the leaflets 805 and/or actuation wires 807 may be biased to extend generally in parallel with the frame 804. In response to signals and/or pulses from one or more actuators and/or controllers, the leaflets 805 and/or actuation wires 807 may be configured to extend into the generally perpendicular and/or closed form with respect to the frame 804 shown in FIG. 8. The one or more actuation wires 807 may be configured to form paddles extending at least partially along the one or more leaflets 805.

One or more actuation wires 807 may be configured to responsive to changes in temperature. For example, the one or more actuation wires 807 may be configured to extend into the closed form in response to temperature at the valve 802 exceeding a threshold temperature value. The actuation wires 807 may be shape set to be responsive to a predetermined threshold temperature. The actuation wires 807 may be set to maintain a closed and/or relaxed position in response to temperatures at or moderately above normal body temperature. In some examples, the one or more actuation wires 807 may be heated with a current to increase the temperature of the actuation wires 807 and/or to cause the one or more actuation wires 807 to extend into the closed form.

In some examples, the one or more actuation wires 807 may be configured to be controlled and/or powered wirelessly via a cloud server at an external location and/or situated remotely from the patient's body. A wireless charger may be used to wirelessly supply power to one or more actuators and/or controllers within the patient's body. Current may be continuously supplied to the one or more actuation wires 807 to maintain a closed state of the leaflets 805.

The valve 802 may comprise various features and/or mechanisms configured to reopen the valve 802 after the leaflets 805 to an at least partially closed form. In one example, the valve 802 may comprise mechanical pull wires (e.g., fingers) configured to contract and/or pull the leaflets 805 back into an open state. In another example, one or more springs (e.g., composed at least partially of Nitinol and/or other shape memory alloys) may be configured to collapse when heated to pull the leaflets 805 back.

In some examples, the valve 802 may comprise one or more latching mechanisms configured to hold the leaflets 805 in one or more at least partially closed forms to reduce an amount of power used by the valve 802. In one example, the valve 802 may comprise one or more pins and/or rotation mechanisms configured to lock the leaflets 805 in a particular position. In another example, one or more magnetic elements may be configured to mate with corresponding magnetic elements attached to the leaflets 805 and/or may be positioned at different locations along the valve 802 to hold the leaflets 805 in particular positions. The valve 802 may comprise multiple types of latching mechanisms, including active and/or passive latching mechanisms. The valve 802 may comprise one or more latching mechanisms configured to quickly release the leaflets 805 in response to signals. For example, in emergency situations, the one or more latching mechanisms may be configured to recede and/or release the leaflets 805.

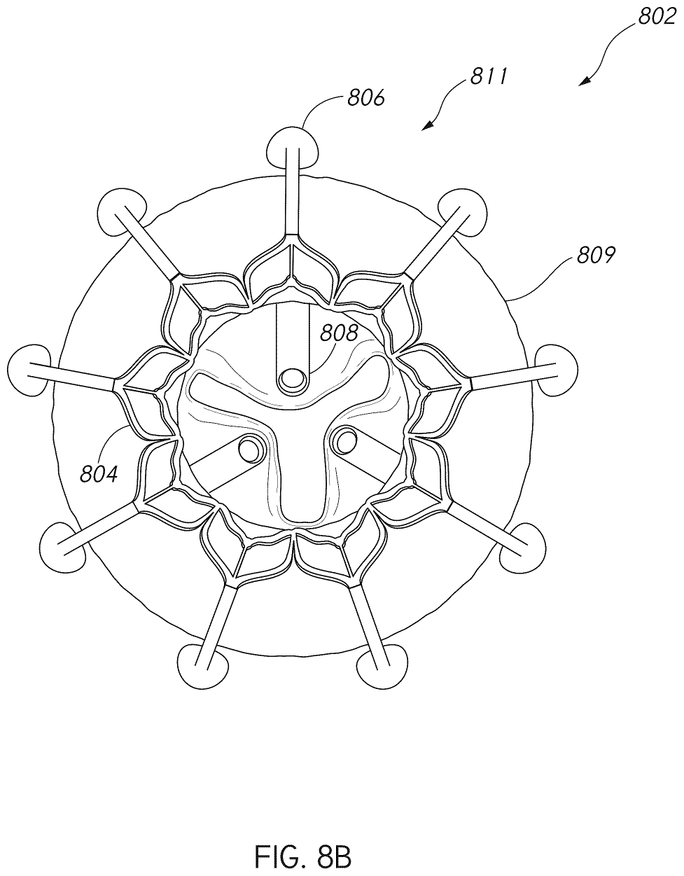

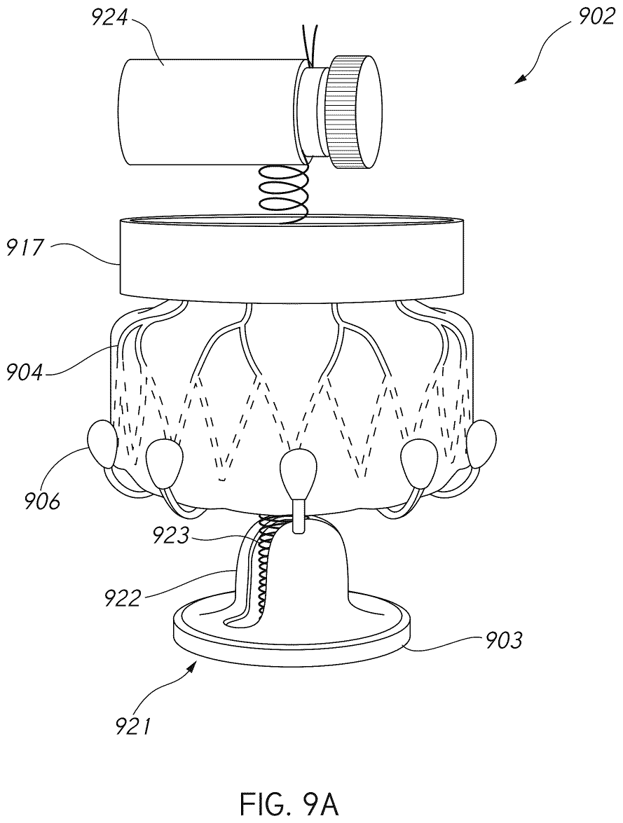

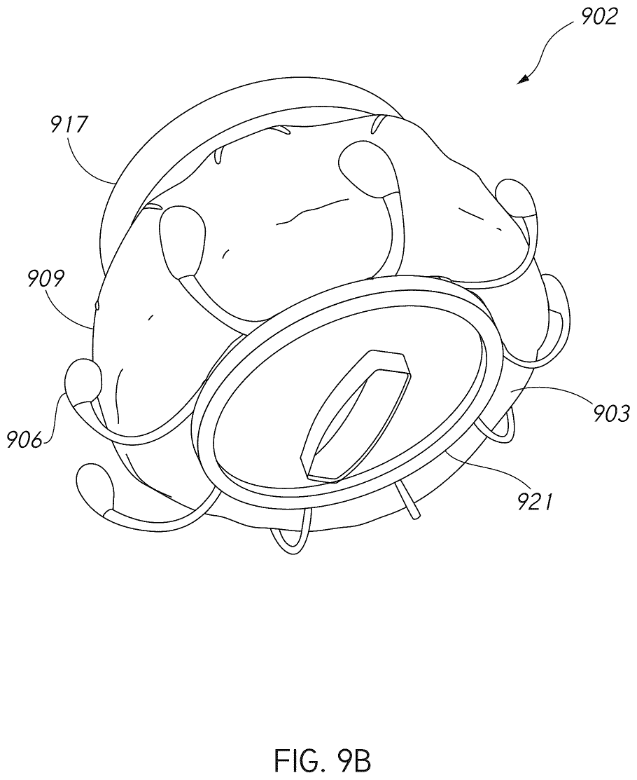

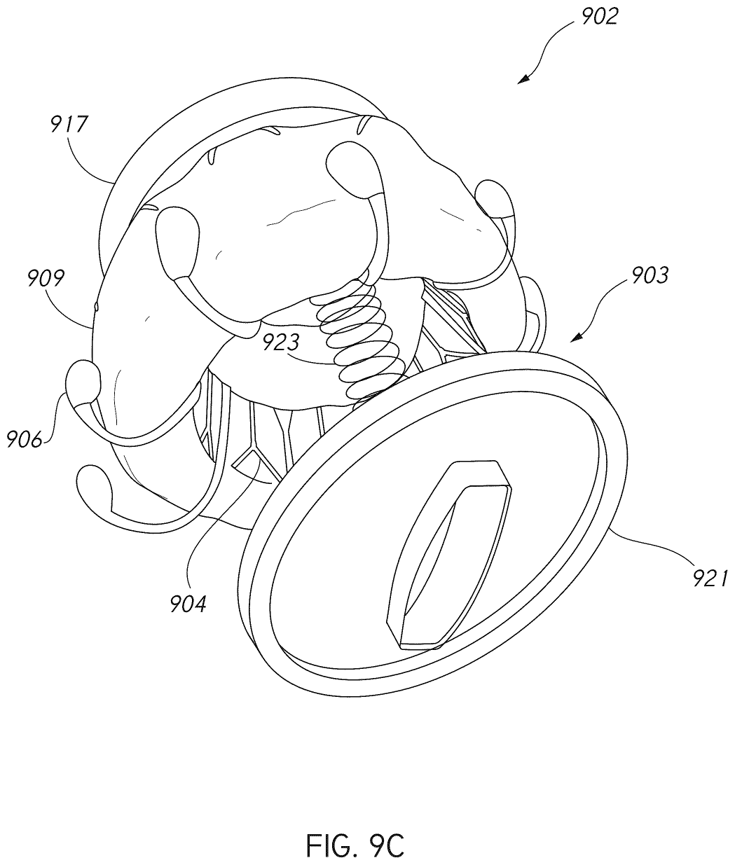

FIGS. 9A-9C illustrate another example occlusion valve 902 configured to dynamically occlude one or more blood vessels of a heart in accordance with one or more examples. FIG. 9A provides a side view of the valve 902 in an at least partially open form. FIG. 9B provides a perspective view of the valve 902 in a closed form. FIG. 9C provides a perspective view of the valve 902 in an at least partially open form.

The valve 902 may comprise one or more leaflets and/or caps 903 configured to extend at least partially across a lumen of the valve 902. For example, the valve 902 and/or a frame 904 of the valve 902 may have a generally cylindrical form extending around a generally cylindrical inner lumen. The inner lumen may be configured to allow blood flow through the valve 902. A cap 903 may be configured to selectively open and/or close the inner lumen of the valve 902.

The valve may comprise a skirt 909 configured to extend at least partially along the frame 904 of the valve 902. The frame 904 may comprise a wire stent comprising a network of struts forming one or more cells. In some examples, the skirt 909 may be configured to extend across one or more cells and/or struts formed by the frame 904. The skirt 909 may be at least partially composed of tissue, cloth, fabric, and/or any suitable material and/or may have a generally flexible structure. The valve 902 may comprise one or more arms 906 configured to extend laterally and/or longitudinally from the valve 902 (e.g., from the cap 903) and/or configured to facilitate anchoring of the valve 902 at one or more target locations.

In some examples, a cap 903 may comprise a generally circular and/or disc-like distal portion 921 and/or a base portion 922. The distal portion 921 may extend from the base portion 922. The base portion 922 may have any suitable form and/or may comprise one or more attachment features (e.g., notches, pegs, cavities, arms, fingers, and/or similar mechanisms) configured to attach to a coil 923 (e.g., spring) and/or similar device (e.g., wire, arm, finger, elongate arm, and/or similar mechanism) configured to dynamically control a position of the cap 903 relative to the frame 904 and/or skirt 909. The coil 923 may be any biasing means and/or means for biasing the cap 903 in one or more positions relative to the frame 904 and/or skirt 909. The coil 923 may be coupled to and/or otherwise controlled by an actuator 924 configured to deliver a current and/or pulse to the coil 923 to cause movement and/or adjustment of the coil 923 and/or of the cap 903.

In some examples, positions of the coil 923 and/or cap 903 may be dynamically adjusted using various components of a modulation system and/or various components of the valve 902. For example, a controller may transmit electronic signals and/or pulses from a battery to the coil 923 and/or cap 903 via one or more actuation wires. The actuation wires and/or coil 923 may be composed of any suitable material(s), including Nitinol and/or other shape memory alloys. In some examples, the actuation wires and/or coil 923 may be at least partially conductive and/or may comprise one or more conductive wires configured to conduct electric signals and/or pulses from one or more controllers and/or batteries to the coil 923 and/or cap 903. An actuation wire and/or coil 923 can comprise any wire, cord, arm, elongate member, and/or similar device configured to conduct electric signals and/or current and/or adjust in response to such signals and/or current.

The coil 923 may be shape set in a desired form. For example, the coil 923 may be configured to form one or more loops and/or coils. The coil 923 may be shape set to a desired form, which may be a generally compressed form and/or a generally expanded form. For examples, the coil 923 may be shape set to a generally expanded form in which loops of the coil 923 are generally elongated and/or form relatively large gaps between each other. In response to heating and/or current at the coil 923, the coil 923 may be configured to compress and/or loops of the coil 923 may be configured to move closer together. The coil 923 may be configured to form a scaffold that closes the cap 903 when current is applied to the coil 923 and/or other conductive wires coupled to the coil 923. To open the valve 902, pressure built up from occluding blood flow through a blood vessel may overcome a resistance of the coil 923 and/or cap 903, causing the coil 923 and/or cap 903 to recede and/or form an opening through the valve 902. Additionally or alternatively, one or more devices and/or mechanisms may be configured to force open the coil 923 and/or cap 903. For example, a current may be supplied and/or removed from the coil 923 to cause the coil 923 to relax and/or press the cap 903 away from the frame 904 and/or skirt 909.

In some examples, the valve 902 may be configured for delivery and/or use at the SVC and/or IVC of a heart. For example, the valve 902 may be disposed inferior to one or more renal veins. The valve 902 may be configured to provide intermittent occlusion of one or more blood vessels, including the SVC and/or IVC.

The one or more coils 923 and/or cap 903 may be configured to be powered via one or more actuators and/or controllers. The one or more coils 923 and/or cap 903 may be coupled to and/or affixed to the frame 904 of the valve 902. In some examples, one or more coils 923 and/or actuators 924 may be disposed within silicone tubes. One or more actuators 924 and/or controllers can be affixed and/or coupled to one or more mounts of the frame 904.

The valve 902 may be configured to selectively modulate blood flow through one or more blood vessels in which the valve 902 may be disposed. In some examples, the valve 902 may be configured to be fully implanted within a blood vessel and/or may not be tethered to a catheter and/or other delivery devices.

One or more coils 923 may be configured to responsive to changes in temperature. For example, the one or more coils 923 may be configured to extend into the closed form in response to temperature at the valve 902 exceeding a threshold temperature value. The coils 923 may be shape set to be responsive to a predetermined threshold temperature. The coils 923 may be set to maintain an open and/or closed position in response to temperatures at or moderately above normal body temperature. In some examples, the one or more coils 923 may be heated with a current to increase the temperature of the coils 923 and/or to cause the one or more coils 923 to extend into the closed and/or open form.

In some examples, the one or more coils 923 may be configured to be controlled and/or powered wirelessly via a cloud server at an external location and/or situated remotely from the patient's body. A wireless charger may be used to wirelessly supply power to one or more actuators and/or controllers within the patient's body. Current may be continuously supplied to the one or more coils 923 to maintain an open and/or closed state of the cap 903.

The valve 902 may comprise various features and/or mechanisms configured to reopen the valve 902 after the cap 903 moves to an at least partially closed form. In one example, the valve 902 may comprise mechanical pull wires (e.g., fingers) configured to contract and/or pull the cap 903 back into an open state. In another example, one or more springs (e.g., composed at least partially of Nitinol and/or other shape memory alloys) may be configured to extend when heated to push the cap 903 away from the frame 904 and/or skirt 909.

In some examples, the valve 902 may comprise one or more latching mechanisms configured to hold the coil 923 and/or cap 903 in one or more at least partially closed forms to reduce an amount of power used by the valve 902. In one example, the valve 902 may comprise one or more pins and/or rotation mechanisms configured to lock the coil 923 and/or cap 903 in a particular position. In another example, one or more magnetic elements may be configured to mate with corresponding magnetic elements attached to the coil 923 and/or cap 903 and/or may be positioned at different locations along the valve 902 to hold the coil 923 and/or cap 903 in particular positions. The valve 902 may comprise multiple types of latching mechanisms, including active and/or passive latching mechanisms. The valve 902 may comprise one or more latching mechanisms configured to quickly release the coil 923 and/or cap 903 in response to signals. For example, in emergency situations, the one or more latching mechanisms may be configured to recede and/or release the coil 923 and/or cap 903.

In some examples, the valve 902 may comprise a ring 917 at a distal end of the valve 902. The ring 917 may have a generally rigid structure and/or may be configured to mount and/or couple to the actuator 924.

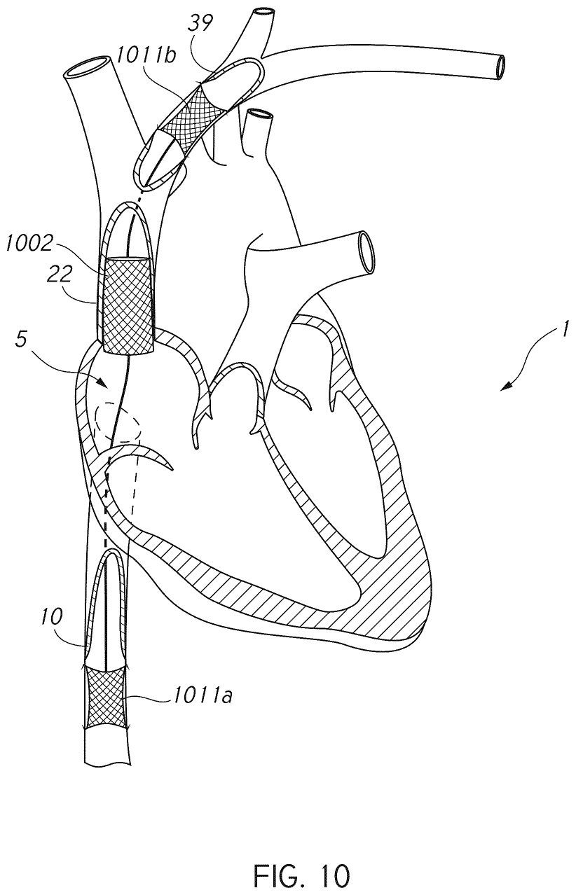

FIG. 10 illustrates an example system comprising one or more valves 1002 in accordance with one or more examples described herein. In some examples, the various valves 1002 described herein may be anchored via one or more anchors 1011, which can include a first anchor 1011a and/or a second anchor 1011b. The one or more anchors 1011 may comprise stents and/or wire frames. In some examples, the valve 1002 and/or one or more anchors 1011 may be self-expandable and/or balloon expandable.

In some examples, the first anchor 1011a may be disposed in a brachiocephalic vein 39 and/or other blood vessels superior to the right atrium 5 (e.g., the SVC 22). The second anchor 1011b may be disposed at least partially within the IVC 10 and/or other blood vessel inferior to the right atrium 5. The first anchor 1011a may extend in a first direction (e.g., distally from the valve 1002) and/or the second anchor 1011b may extend in a second direction (e.g., proximally from the valve 1002).

The valve 1002 may have any suitable form and/or may comprise any of the valves 1002 described herein. In some examples, the valve 1002 may have a barbed form. The valve 1002 may comprise a single frame or multiple frames.

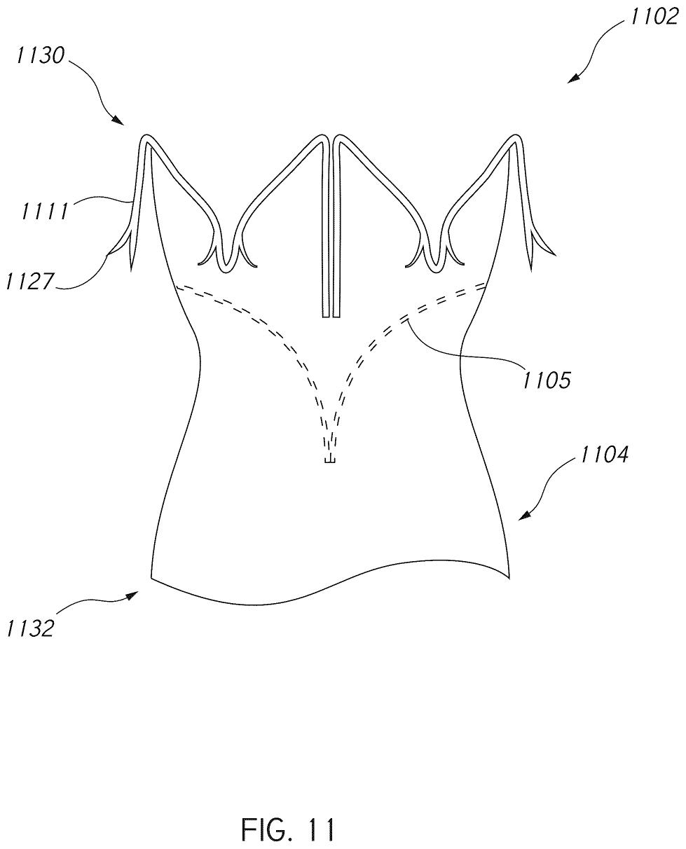

FIG. 11 illustrates an example frame 1104 of an occlusion valve 1102 described in any of the examples herein. The frame 1104 may be a passive flow restriction device and/or may be comprised of a balloon-expandable and/or self-expandable wire form and/or stent. In some examples, the frame 1104 may be constructed in a shape to reduce flow through the frame 1104. For example, the frame 1104 may be generally curved and/or may have an hourglass shape.

In some examples, the frame 1104 may comprise one or more barbs 1127 at a distal end 1130 and/or proximal end 1132 of the frame 1104. For example, the frame 1104 may comprise a flange 1111 at the distal end 1130 and/or the one or more barbs 1127 may be configured to improve anchoring of the flange 1111 and/or frame 1104. The one or more barbs 1127 can include any suitable anchoring means and/or means for anchoring, which can include spikes, arms, fingers, protrusions, needles, pincers, and/or similar devices.

The frame 1104 may be configured to operate passively and/or may incorporate one or more active elements that can allow variable adjustment of an amount of flow restriction of the frame 1104 and/or other components of an example valve (e.g., leaflets and/or caps). An example active element may include Nitinol actuation wires and/or other suitable devices. In some examples, the frame 1104 may be incorporated within one or more ratcheting mechanisms to dynamically adjust one or more actuation features (e.g., actuation wires) of the valve and/or frame 1104. The ratcheting mechanism may be configured to dynamically adjust an amount of flow restriction to a fixed measurement and/or retain a given position and/or measurement without sustained active activation.

The frame 1104 and/or various valves described herein may be suitable for dynamically adjusting an amount of flow restriction despite having a uniform and/or constant surface area and/or size. For example, physicians can advantageously utilize a single valve size and/or form for multiple patients and/or for causing multiple flow effects. In addition, patients may not require extensive hemodynamic testing prior to procedures to determine which size of implant to use.

The valve 1102 may comprise one or more leaflets 1105 configured to extend at least partially across an inner lumen of the frame 1104. The valve 1102 may comprise any number of leaflets 1105 (e.g., two or three leaflets 1105).

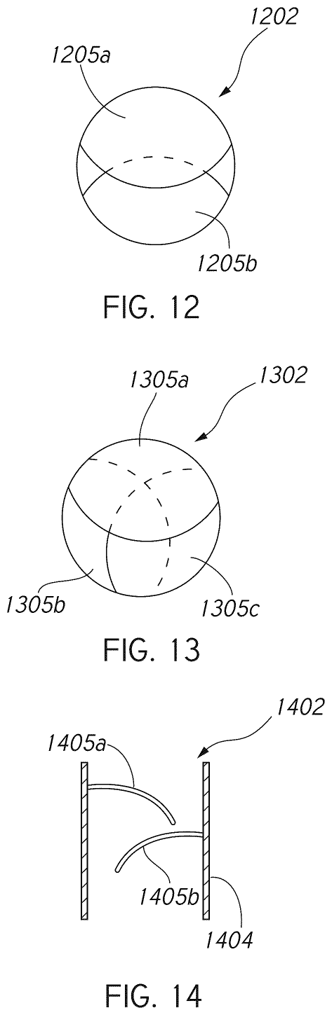

FIG. 12 provides an overhead view of a valve 1202 comprising multiple leaflets 1205 in accordance with one or more examples described herein. The valve 1202 illustrated in FIG. 12 comprises two leaflets 1205 in a closed form (e.g., extending fully or approximately fully over a lumen of the valve 1202). However, example valves 1202 can comprise other numbers of leaflets 1205 (e.g., one, three, or four). The valve 1202 comprises a first leaflet 1205a and a second leaflet 1205b. The first leaflet 1205a and the second leaflet 1205b may at least partially overlap in one or more partially closed forms and/or in the fully closed form.

The one or more leaflets 1205 may be dynamically adjustable and/or may be configured to controllably and/or passively move between one or more positions, including partially closed positions, fully open positions, and/or fully closed positions. The valve 1202 may comprise one or more latching mechanisms configured to hold the leaflets 1205 in one or more positions.

In some examples, the leaflets 1205 may have generally curved forms. For example, one or more leaflets 1205 may have generally ovular and/or partial circular shapes.

FIG. 13 provides an overhead view of a valve 1302 comprising multiple leaflets 1305 in accordance with one or more examples described herein. The valve 1302 illustrated in FIG. 13 comprises three leaflets 1305 in a closed form (e.g., extending fully or approximately fully over a lumen of the valve 1302). However, example valves 1302 can comprise other numbers of leaflets 1305 (e.g., one, two, or four). The valve 1302 comprises a first leaflet 1305a, a second leaflet 1305b, and/or a third leaflet 1305c. The first leaflet 1305a, the second leaflet 1305b, and/or the third leaflet 1305c may at least partially overlap in one or more partially closed forms and/or in the fully closed form.

The one or more leaflets 1305 may be dynamically adjustable and/or may be configured to controllably and/or passively move between one or more positions, including partially closed positions, fully open positions, and/or fully closed positions. The valve 1302 may comprise one or more latching mechanisms configured to hold the leaflets 1305 in one or more positions.

In some examples, the leaflets 1305 may have generally curved forms. For example, one or more leaflets 1305 may have generally ovular and/or partial circular shapes.

FIG. 14 provides a side view of a valve 1402 comprising multiple leaflets 1405 extending from a frame 1404 in accordance with one or more examples described herein. The valve 1402 illustrated in FIG. 14 comprises two leaflets 1405 in a closed form (e.g., extending fully or approximately fully over a lumen of the valve 1402. However, example valves 1402 can comprise other numbers of leaflets 1405 (e.g., one, three, or four). The valve 1402 comprises a first leaflet 1405a and a second leaflet 1405b. The first leaflet 1405a and the second leaflet 1405b may at least partially overlap in one or more partially closed forms and/or in the fully closed form.

The one or more leaflets 1405 may be dynamically adjustable and/or may be configured to controllably and/or passively move between one or more positions, including partially closed positions, fully open positions, and/or fully closed positions. The valve 1402 may comprise one or more latching mechanisms configured to hold the leaflets 1405 in one or more positions.

In some examples, the leaflets 1405 may have generally curved forms. For example, one or more leaflets 1405 may have generally ovular and/or partial circular shapes.

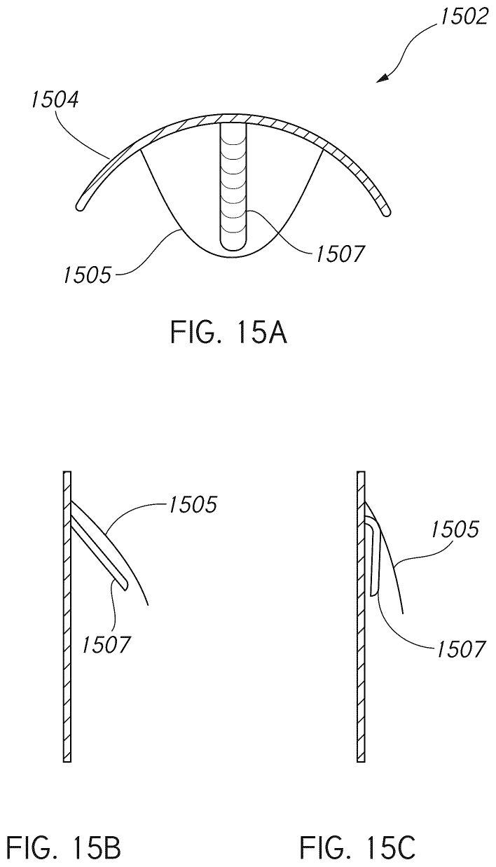

FIGS. 15A-15C illustrate an example valve 1502 and/or one or more components of a valve 1502 in accordance with one or more examples described herein. FIG. 15A provides an overhead view of a leaflet 1505 of the valve 1502 and/or a support arm 1507 extending at least partially along the leaflet 1505. FIG. 15B provides a side view of the leaflet 1505 with the support arm 1507 in an expanded and/or rigid form, holding the leaflet 1505 away from a frame 1504 of the valve 1502 and/or extending the leaflet 1505 at least partially over a lumen of the valve 1502. FIG. 15C provides a side view of the leaflet 1505 with the support arm 1507 in a collapsed and/or flexible form, allowing the leaflet 1505 to drape along the frame 1504 and/or not occluding the lumen of the valve 1502. The support arm 1507 may be collapsible and/or may be configured to collapse (e.g., break, bend, and/or sag) and/or stiffen in response to signals and/or changes in blood pressure.

The support arm 1507 may be any supporting means and/or means for supporting the leaflet 1505, and may include actuation wires and/or other actuation members, elongate arms, fingers, and/or similar devices. In some examples, the support arm 1507 may be configured to adjust and/or transform between rigid and/or flexible forms. For example, the support arm 1507 may have characteristics similar to a tape measure, in which the support arm 1507 has a generally curved lateral form. The support arm 1507 may be configured to assume a generally rigid form (e.g., in response to a current provided to the support arm 1507. In some examples, the support arm 1507 may be configured to be responsive to increasing pressure and/or may be configured to collapse and/or “crack” in response to increased pressure. For example, increased pressure may cause the support arm 1507 and/or a portion of the support arm 1507 to bend, which may impact the structural integrity of the support arm 1507 and/or release a tension holding the support arm 1507 in the straight and/or elongate form. In some examples, the support arm 1507 may operative passively and/or may be configured to adjust between the rigid and flexible forms in response to blood flow changes and/or may not require an external stimulus (e.g., a current).

The valve 1502 may comprise additional leaflets 1505 and/or support arms 1507 not shown in FIGS. 15A-15C. For example, the valve 1502 may comprise two or three leaflets 1505 and/or two or three support arms 1507.

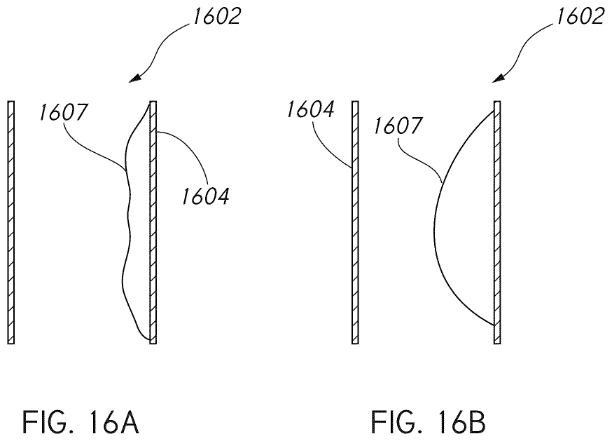

FIGS. 16A and 16B illustrate an example occlusion valve 1602, which may comprise various component of valves 1602 described herein in accordance with one or more examples. The valve 1602 may comprise a frame 1604 and/or one or more leaflets, caps, biasing members, and/or actuation members as described in other examples herein. In some examples, the valve 1602 may comprise an occlusion element 1607, which can comprise any occlusion means and/or means for occluding a lumen of the valve 1602, and can include one or more balloons, diaphragms, leaflets, and/or similar devices. The occlusion element 1607 may be configured to expand and/or inflate passively and/or in response to an external stimulus (e.g., a current). FIG. 16A illustrates the valve 1602 in an open and/or unexpanded form in which the occlusion element 1607 does not extend and/or minimally extends across the lumen of the valve 1602. FIG. 16B illustrates the valve 1602 in a closed and/or expanded form in which the occlusion element 1607 extends at least partially across the lumen of the valve 1602.

The occlusion element 1607 may be coupled to and/or affixed to the frame 1604 and/or may be configured to expand away from the frame 1604 in response to blood flow changes and/or external stimuli. In some examples, as increased blood volume presses against the occlusion element 1607 in the expanded form, the occlusion element 1607 may be configured to responsively recede and/or open the lumen in response to the blood flow. In some examples, the occlusion element 1607 may be coupled to and/or extend from an inner surface of the frame 1604 and/or may be configured to extend across and/or at least partially occlude an inner lumen of the frame 1604 as the occlusion element 1607 inflates.

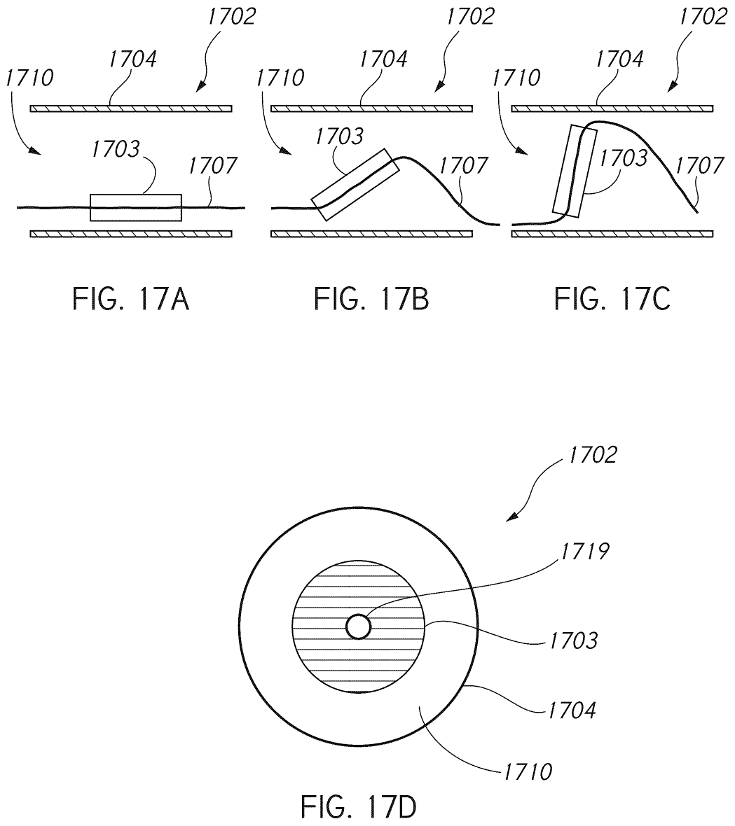

FIGS. 17A-17D illustrate an example valve 1702 configured to dynamically occlude one or more blood vessels of a heart in accordance with one or more examples herein. The valve 1702 may comprise a frame 1704 and/or one or more leaflets and/or occlusion elements described in other examples herein. In some examples, the valve 1702 may comprise a cap 1703 having a circular and/or disc shape. FIG. 17A provides a side view of the cap 1703 in an open state in which a major (e.g., circular) axis of the cap 1703 extends generally in parallel with walls of the frame 1704. The cap 1703 may be configured to assume the open state by default and/or may present minimal blockage of the lumen 1710 while in the open state. FIG. 17B provides a side view of the cap 1703 in a partially closed state in which the major axis of the cap 1703 extends at an angle relative to the walls of the frame 1704. FIG. 17C provides a side view of the cap 1703 in a generally closed state in which the major axis of the cap 1703 extends generally perpendicularly to the walls of the stent 1704. FIG. 17D provide a top view of the valve 1702 in the closed state. While the cap 1703 is shown having a smaller diameter than the frame 1704 in FIG. 17D, the cap 1703 may have a diameter approximately equal to the frame 1704 and/or may be configured to more fully close a lumen 1710 of the valve 1702. The cap 1703 may be at least partially and/or fully disposed within the lumen 1710 of the valve 1702.

In some examples, the valve 1702 may comprise an actuation element 1707 (e.g., wire) configured to extend at least partially through the cap 1703. The actuation element 1707 may extend through a length of the cap 1703. The actuation element 1707 may be configured to extend through an aperture 1719 at or near a midpoint of the cap 1703. The aperture 1719 may be disposed at a central portion of the cap 1703. In some examples, the actuation element 1707 may have shape memory characteristics and/or may be at least partially composed of Nitinol and/or other shape memory alloy. The actuation element 1707 may be configured to bend in response to a current and/or other stimulus from a controller and/or battery. Bending of the actuation element 1707 may be configured to cause rotation of the cap 1703 from the open state to the partially closed and/or fully closed states. In some examples, the cap 1703 may have multiple partially closed states. The valve 1702 may comprise one or more latching mechanisms configured to hold the cap 1703 in the one or more partially closed states, the open state, and/or the fully closed state.

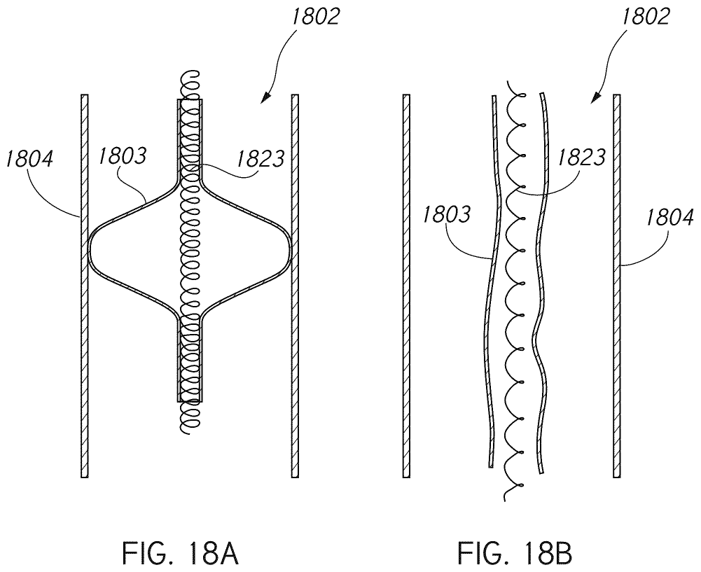

FIGS. 18A and 18B illustrate an example valve 1802 configured to dynamically occlude one or more blood vessels of a heart in accordance with one or more examples herein. The valve 1802 may comprise a coil 1823 (e.g., biasing element) and/or occlusion element 1803 (e.g., diaphragm) configured to dynamically close the lumen of the valve 1802. FIG. 18A provides a side view of the coil 1823 and/or occlusion element 1803 in an at least partially closed state. FIG. 18B provides a side view of the coil 1823 and/or occlusion element 1803 in an open state. The coil 1823 and/or occlusion element 1803 may be configured to assume the stretched and/or open state shown in FIG. 18B by default and/or may compress to the form shown in FIG. 18A in response to a signal and/or current at and/or through the coil 1823.

The coil 1823 and/or occlusion element 1803 (e.g., covering) may extend at least partially through a frame 1804 of the valve 1802. The coil 1823 and/or occlusion element 1803 may extend at least partially through and/or along a lumen formed by the frame 1804 of the valve 1802.

The coil 1823 may comprise an actuation element configured to extend at least partially through the occlusion element 1803. In some examples, the occlusion element 1803 may be configured to at least partially enclose at least a portion of the coil 1823. The coil 1823 may be responsive to currents and/or other stimuli provided to the coil 1823 via a controller and/or battery. In some examples, the occlusion element 1803 may have shape memory characteristics and/or may be at least partially composed of Nitinol and/or other shape memory alloy.

In some examples, the coil 1823 and/or occlusion element 1803 may be biased towards the open (e.g., stretched) form shown in FIG. 18B, in which loops of the coil 1823 may be expanded and/or the occlusion element 1803 may be stretched to a relatively high length. The occlusion element 1803 may have a generally flexible and/or elastic structure and/or may be at least partially composed of fabric, polymers, and/or similar materials. In response to a current, the coil 1823 may be configured to compress, which may cause the occlusion element 1803 to compress and/or expand laterally, as shown in FIG. 18A. Lateral expansion of the occlusion element 1803 may cause occlusion of the lumen of the valve 1802. In the expanded form shown in FIG. 18A, the occlusion element 1803 may assume a circular and/or disc shape at or near a midsection of the occlusion element 1803.

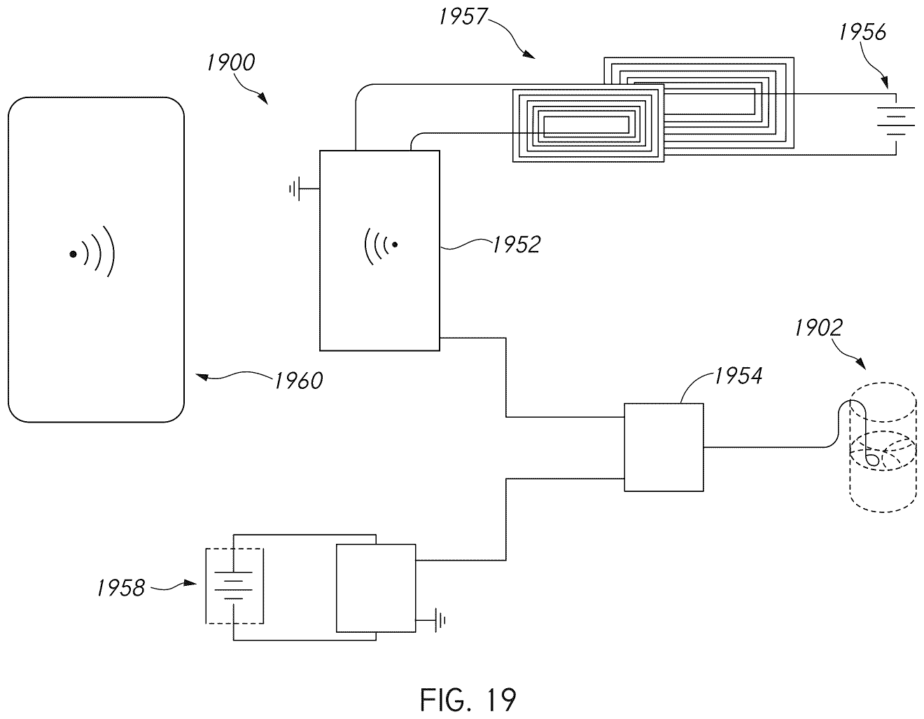

FIG. 19 illustrates an example circuit 1900 configured to control and/or power one or more valves 1902 described in examples herein. The circuit 1900 may be an implantable wireless circuit configured to control the various valves described herein. The circuit 1900 may comprise a microcontroller 1952. The microcontroller 1952 may utilize native Bluetooth Low Energy (BLE) and/or WIFI chipsets. The circuit 1900 may further comprise an integrated circuit 1954 utilizing voltage regulators and/or transistors to modulate power delivery as indicated by the output signals from the microcontroller 1952. The circuit 1900 may comprise multiple power sources, which can include a low energy power source 1956 supplied by a wireless induction coil 1957 (e.g., transceiver) to power the microcontroller 1952 and/or a high energy power source 1958 constructed of a wirelessly rechargeable battery which can supply power to the valve 1902. Furthermore, the BLE and/or WIFI functionalities of the microcontroller 1952 can allow for wireless communication from the microcontroller 1952 to a central device 1960 (e.g., a computer, mobile device, cloud server, etc.). Further encryption of the wireless communications may be constructed via Advanced Encryption Standard (AES) and/or end-to-end encryption protocols.

The circuit 1900 may utilize a closed-loop feedback system to control the valve 1902. The circuit 1900 may initiate in a sensing mode, where pressure sensors inferior and/or superior to the valve 1902 (e.g., located in the right atrium and/or caval vessel) may be used to monitor pressures and/or gradients across the valve 1902 and/or send data back to the controller 1952. The controller can then calculate optimal filling pressures and/or a required current to open, close, and/or partially close the valve 1902. The circuit 1900 can switch into an activation mode in which the controller 1952 can adjust the valve 1902 to the previously calculated values to achieve optimal filling pressures. The circuit 1900 can then switch back to sensing mode to begin the closed-loop cycle again. Additionally, the controller 1952 may have its power supplied via wireless induction or an implantable battery. The controller 1952 may also have a central device 1960 outside the patient used to transmit data to and/or from the controller 1952 and/or may be used to monitor the device and its critical care sensors. This data then can be uploaded to the patient's health care provider to allow for long term data acquisition and device management.

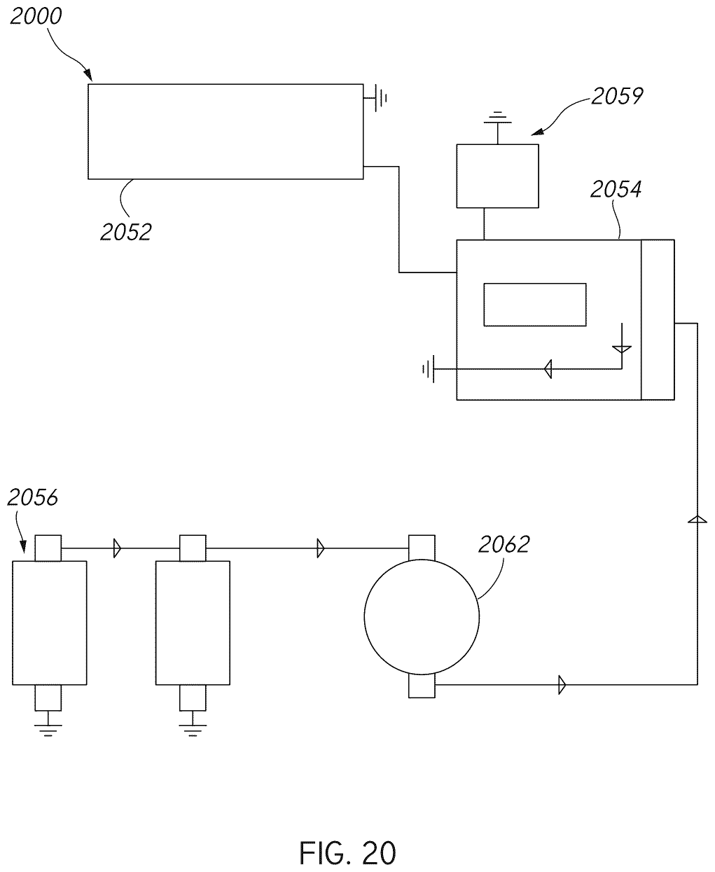

FIG. 20 illustrates another circuit 2000 for controlling one or more valves described herein in accordance with one or more examples. The circuit 2000 may comprise a controller 2052 (e.g., microcontroller), an integrated circuit 2054, a power source 2056, and/or an actuator 2062 configured to dynamically control one or more valves. The circuit 2000 may further comprise a pull-down resistor 2059 coupled to the integrated circuit 2054.

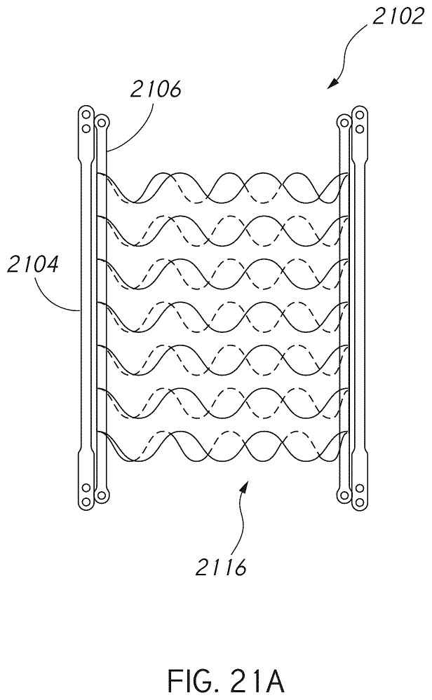

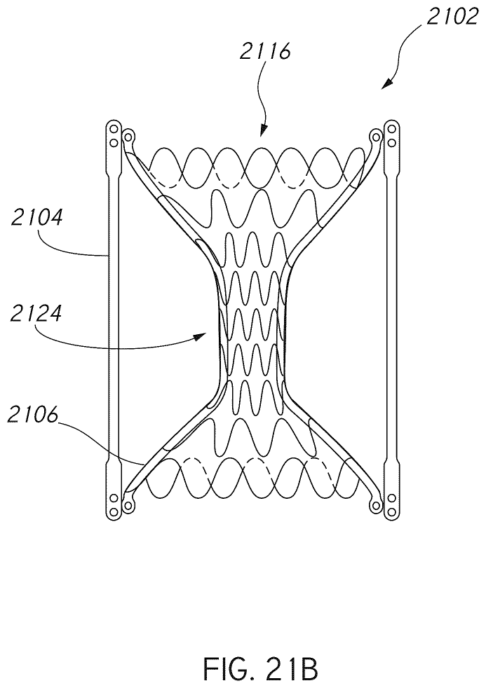

FIGS. 21A and 21B illustrate an example dynamic occlusion valve 2102 configured for placement at least partially within one or more blood vessels of a heart in accordance with one or more examples herein. FIG. 21A provides a side view of the valve 2102 in an open state and FIG. 21B provides a side view of the valve 2102 in a closed state.

The valve 2102 may comprise an outer frame 2104 and/or an inner frame 2106. The outer frame 2104 and/or inner frame 2106 may comprise networks of struts forming one or more cells through the outer frame 2104 and/or inner frame 2106. In some examples, the inner frame 2106 may have a generally solid structure and/or may comprise a covering configured to cover one or more cells of the inner frame 2106.

The outer frame 2104 and/or inner frame 2106 may be at least partially composed of Nitinol and/or other shape memory alloys. In some examples, the outer frame 2104 and/or inner frame may have generally elastic and/or super elastic structures. The outer frame 2104 and/or inner frame may have shape memory characteristics and/or may be configured to naturally assume a given form. In some examples, the inner frame 2106 may be configured to naturally assume an expanded form shown in FIG. 21A and/or to naturally constrict and/or reduce in diameter in response to changes in temperature and/or blood flow. The inner frame 2106 may be configured to constrict to form a generally narrow waist 2124 at or near a midsection of the inner frame 2106. The inner frame may assume an hourglass shape in response to a signal and/or current at the valve 2102.

The valve 2102 may comprise a network of struts 2116, which can include thin metallic wires and/or similar materials. The struts 2116 may be coupled to the inner frame 2106 and/or may extend between arms and/or spines of the inner frame 2106. The struts 2116 may have generally curved and/or coiled forms and/or may be configured to compress (e.g., bend) and/or expand (e.g., straighten) in response to pressure and/or current at and/or through the valve 2102 (e.g., through the struts 2116). In some examples, the struts 2116 may be configured to cause bending of the inner frame 2106 and/or the inner frame 2106 may be configured to cause compression of the struts 2116. The inner frame 2106 may comprise generally straight arms in a default form.

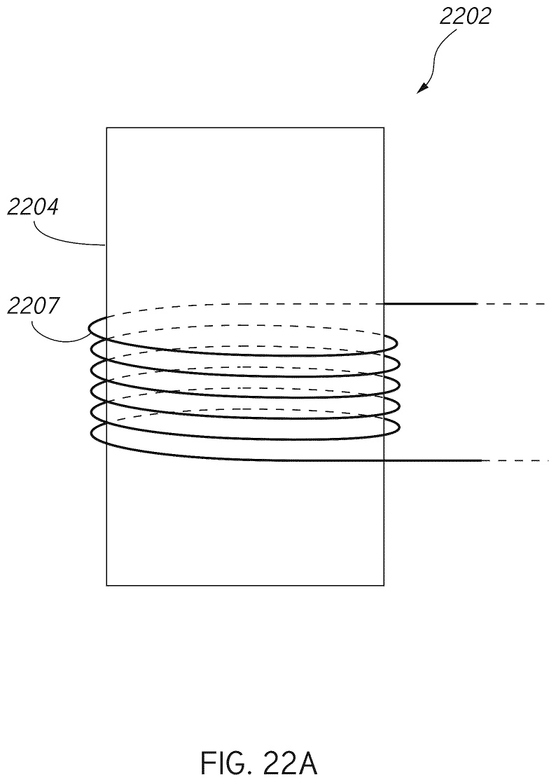

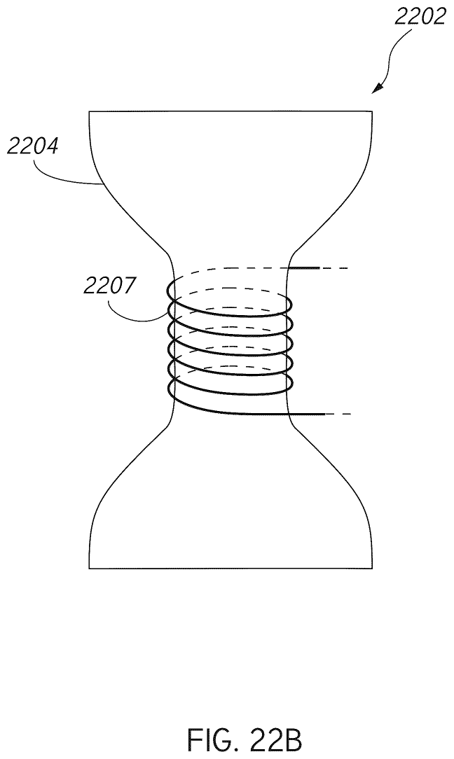

FIGS. 22A and 22B illustrate another example valve 2202 configured to dynamically occlude one or more blood vessels of a heart in accordance with one or more examples. FIG. 22A illustrates the valve 2202 in an open form and FIG. 22B illustrates the valve 2202 in a closed form. The valve 2202 may comprise a frame 2204 shape set to a generally cylindrical and/or tubular form. In some examples, the valve 2202 may further comprise an actuation wire 2207 and/or coil around the frame 2204 and/or configured to at least partially enclose the frame 2204. The actuation wire 2207 may be at least partially composed of Nitinol and/or other shape memory alloys and/or may be wrapped in loops around at least a midsection of the tubular frame 2204. When current is applied to the actuation wire 2207, the actuation wire 2207 may be configured to compress and/or shrink in length and/or diameter, causing the coils and/or at least the midsection of the frame 2204 to reduce in diameter. This reduction in inner diameter of the frame 2204 can result in a reduction in flow through the valve 2202. The frame 2204 may assume an hourglass shape in response to the current at the wire 2207.

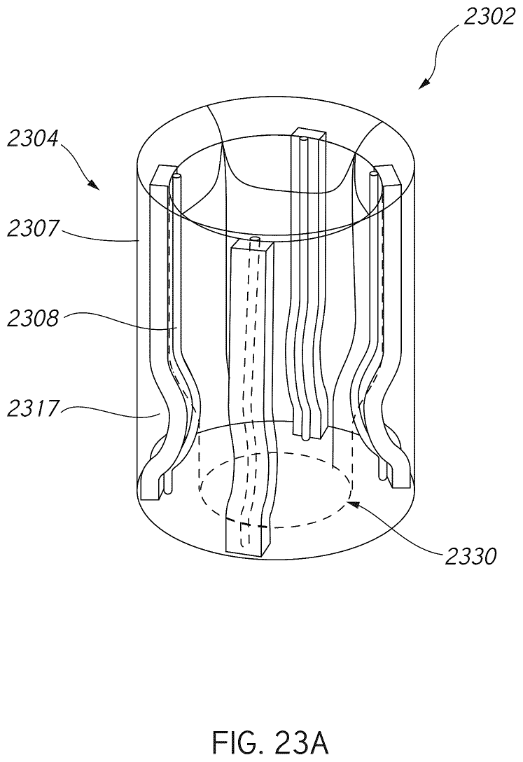

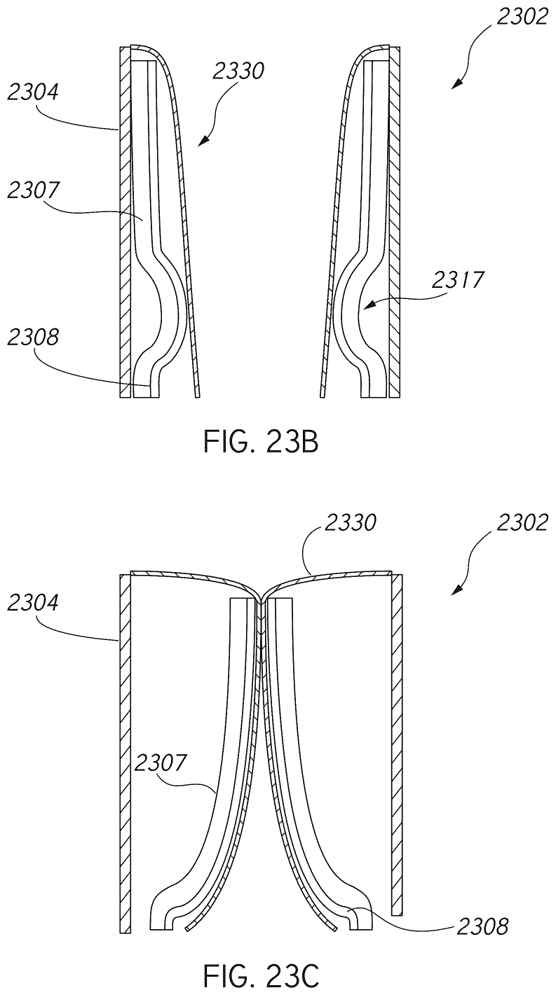

FIGS. 23A-23C illustrate another example valve 2302 configured to dynamically occlude one or more blood vessels of a heart in accordance with one or more examples herein. FIG. 23A provides a perspective view of the valve 2302 in an open state. FIG. 23B provides a side view of the valve 2302 in the open state. FIG. 23C provides a side view of the valve 2302 in a closed state, in which one or more actuation members 2307 press an occlusion sheet 2330 (e.g., one or more leaflets) over a lumen of the valve 2302. The one or more actuation members 2307 may be configured to extend inwardly in response to current and/or signals at actuation wires 2308 coupled to the actuation members 2307.

In some examples, an actuation member 2307 can comprise a composite cantilever beam configured to extend longitudinally along a frame 2304 of the valve 2302. The actuation members 2307 can comprise actuation wires 2308 (e.g., composed of Nitinol) extending along the actuation members 2307 and/or elongate columns of the valve 2302. The actuation wires 2308 may be embedded into the sheet 2330, which can comprise substrates (e.g., tissue and/or synthetic leaflets) to cause deflection of the sheet 2330.

In some examples, the actuation members 2307 may comprise one or more inward and/or outward bends 2317 to allow for increased closing force on the sheet 2330 and/or to allow for using occluding flow to force the sheet 2330 into the closed form shown in FIG. 23C. A bend 2317 may form an inward bulge at an actuation member 2307.

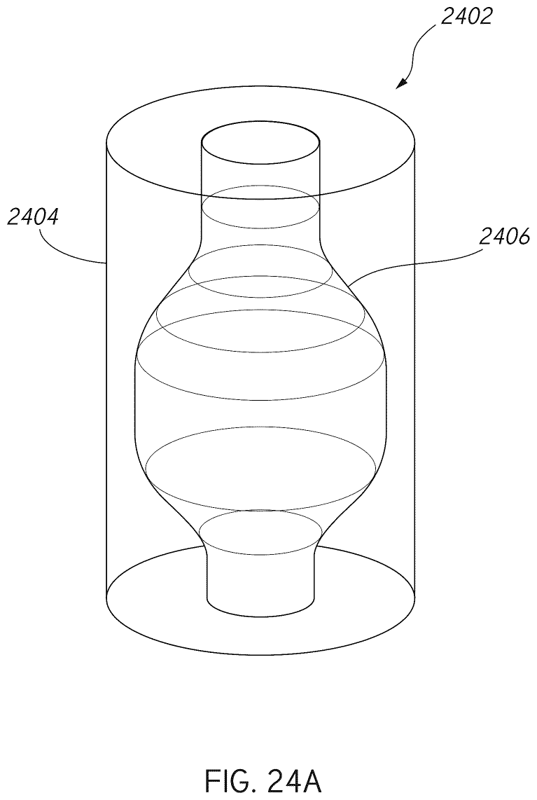

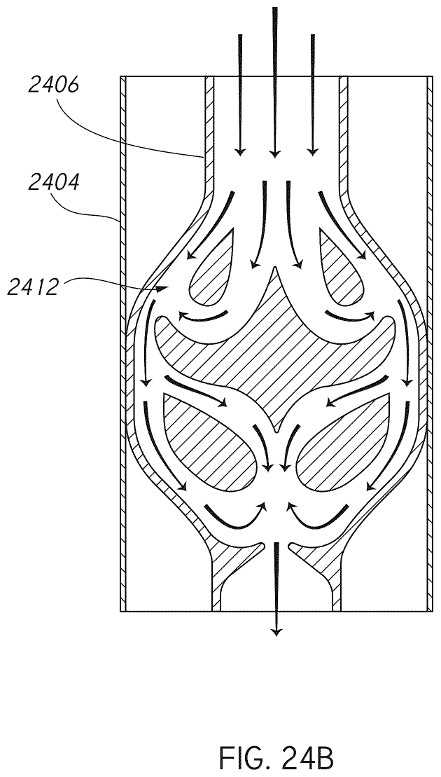

FIGS. 24A and 24B illustrate another example valve 2402 configured to dynamically occlude one or more blood vessels of a heart in accordance with one or more examples. FIG. 24A provides a first cross section of the valve 2402 showing an inner frame 2406 of the valve 2402. FIG. 24B provides a second cross section of the valve 2402 showing pathways 2412 built into the outer frame 2404 of the valve 2402. The outer frame 2404 and/or inner frame 2406 may be constructed similarly to a tesla valve, in which flow through the valve 2402 may be slowed due to the various pathways 2412 of the valve 2402.

Flow through the valve 2402 may be selectively and/or dynamically directed either through a generally open inner frame 2406 of the valve 2402 or through the pathways 2412 of the outer frame 2404. For example, one or more caps, leaflets, check valves, and/or other occlusion elements may be configured to selectively close a path through the inner frame 2406 of the valve 2402. In such cases, flow may be force through the pathways 2412 of the outer frame 2404, thus limiting flow through the blood vessel. The pathways 2412 may have a three-dimensional form and/or may have any suitable structure. In some examples, the valve 2402 may comprise multiple pathways 2412, with various pathways, having upward curves and/or downward curves to slow and/or impede flow through the valve 2402.

The valve 2402 may comprise one or more stoppers configured to selectively block blood flow through pathways other than the curved pathways 2412. For example, one or more stoppers may be configured to prevent blood flow between the outer frame 2404 and the inner frame 2406 to force blood flow through the inner frame 2406 and/or through the curved pathways 2412. In some examples, signals and/or currents at the valve 2402 may be configured to activate stoppers at the valve 2402.

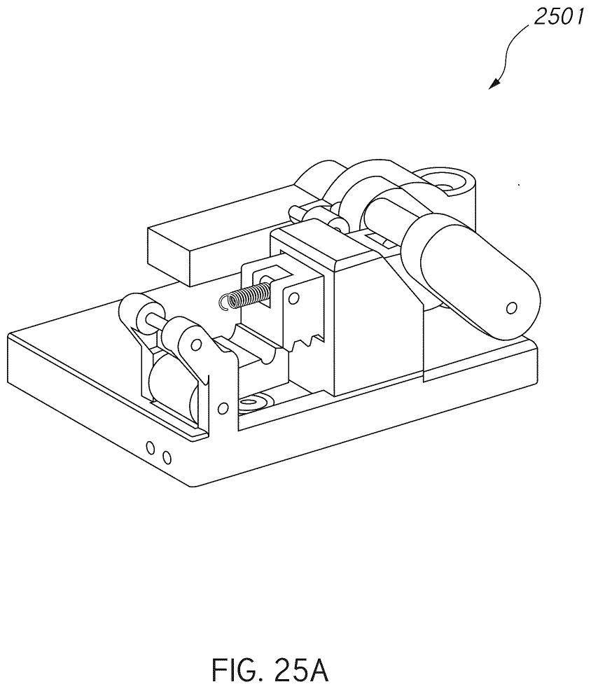

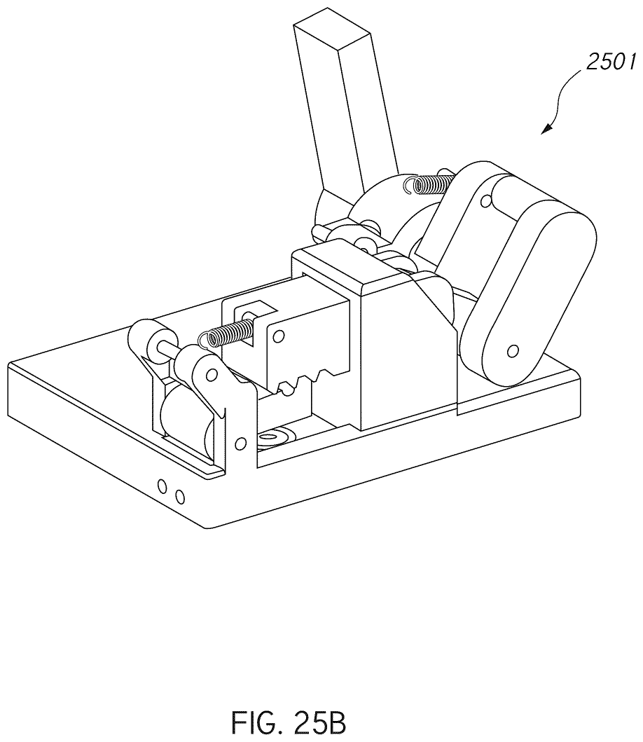

FIGS. 25A and 25B illustrate an example ratcheting mechanism 2501 configured to allow for dynamic adjustments of one or more components of various valves described herein. The ratcheting mechanism 2501 may be configured to vary flow restriction in fixed increments, without requiring sustained activation of one or more deflection mechanisms, thereby reducing total energy consumption and power requirements.

In some examples, a frame of a valve may be incorporated with one or more ratcheting mechanisms 2501 to dynamically adjust one or more actuation features (e.g., actuation wires) of the valve and/or frame. The ratcheting mechanism 2501 may be configured to dynamically adjust an amount of flow restriction to a fixed measurement and/or retain a given position and/or measurement without sustained active activation.

Described herein are various example medical implants and/or delivery methods. Some examples described herein may be used in combination and/or may be used independently.

### Additional Description of Examples

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1: A system for regulating blood flow through a blood vessel of a heart, the system comprising a valve comprising an outer frame and configured for placement at least partially within the blood vessel.

Example 2: The system of any example herein, in particular example 1, further comprising a controller disposed outside the heart, the controller configured to transmit signals to the valve.

Example 3: The system of any example herein, in particular example 2, further comprising a battery configured to supply power to the controller or valve.

Example 4: The system of any example herein, in particular example 2, further comprising a sensor configured for placement within the heart, the sensor configured to transmit signals relating to blood flow to the controller.

Example 5: The system of any example herein, in particular example 4, wherein the controller is configured to transmit signals to the valve based on the signals received from the sensor.

Example 6: The system of any example herein, in particular example 1, wherein the valve comprises two or more leaflets.

Example 7: The system of any example herein, in particular example 6, wherein the two or more leaflets at least partially overlap.

Example 8: The system of any example herein, in particular example 6, further comprising one or more actuation wires configured to control movement of the two or more leaflets.

Example 9: The system of any example herein, in particular example 8, wherein the one or more actuation wires are configured to receive signals from a controller disposed outside of the heart.

Example 10: The system of any example herein, in particular example 8, wherein the one or more actuation wires are configured to cause independent movement of each of the two or more leaflets.

Example 11: The system of any example herein, in particular example 8, wherein the one or more actuation wires are configured to cause simultaneous movement of each of the two or more leaflets.

Example 12: The system of any example herein, in particular example 8, wherein the one or more actuation wires are configured to form loops under or above the two or more leaflets.

Example 13: The system of any example herein, in particular example 12, wherein the loops are configured to press or pull the two or more leaflets from a default open position to one or more at least partially closed positions.

Example 14: The system of any example herein, in particular example 8, wherein the one or more actuation wires are configured to move the two or more leaflets between multiple predetermined positions.

Example 15: The system of any example herein, in particular example 8, wherein the one or more actuation wires are configured to control one or more support arms coupled to the two or more leaflets.

Example 16: The system of any example herein, in particular example 15, wherein the one or more support arms extend along undersides of the two or more leaflets.

Example 17: The system of any example herein, in particular example 1, wherein the valve comprises a cap configured to at least partially occlude a lumen of the outer frame.

Example 18: The system of any example herein, in particular example 17, wherein the cap comprises a disc-shaped distal portion.

Example 19: The system of any example herein, in particular example 17, further comprising one or more actuation wires configured to control an amount of separation between the outer frame and the cap.

Example 20: The system of any example herein, in particular example 19, wherein the one or more actuation wires are configured to hold the cap away from the outer frame in a default and position and wherein the one or more actuation wires are configured to pull the cap towards the outer frame in response to a current through the one or more actuation wires.

Example 21: The system of any example herein, in particular example 19, wherein the one or more actuation wires are coupled to an actuator.

Example 22: The system of any example herein, in particular example 19, wherein the one or more actuation wires form a coiled spring.

Example 23: The system of any example herein, in particular example 17, wherein the cap is disposed at an inflow portion of the valve.

Example 24: The system of any example herein, in particular example 1, further comprising a first anchor tethered to the valve and extending proximally from the valve.

Example 25: The system of any example herein, in particular example 24, further comprising a second anchor tethered to the valve and extending proximally from the valve.

Example 26: The system of any example herein, in particular example 1, wherein the outer frame comprises one or more barbed arms configured to anchor the valve within the blood vessel.

Example 27: The system of any example herein, in particular example 1, further comprising one or more leaflets extending across a lumen of the outer frame and collapsible support arms extending along each of the one or more leaflets.

Example 28: The system of any example herein, in particular example 27, wherein the collapsible support arms are configured to have a flexible default form and are configured to stiffen in response to a current.

Example 29: The system of any example herein, in particular example 1, further comprising a balloon coupled to an inner surface of the outer frame, the balloon configured to have a deflated default form and configured to be inflated with a gas or fluid to at least partially occlude a lumen of the outer frame.

Example 30: The system of any example herein, in particular example 1, further comprising a cap disposed within a lumen of the valve and an actuation wire coupled to the cap.

Example 31: The system of any example herein, in particular example 30, wherein the cap is configured to provide minimal blockage of the lumen in a default form of the cap and the actuation wire.

Example 32: The system of any example herein, in particular example 31, wherein the actuation wire is configured to cause rotation of the cap to increase blockage of the lumen.

Example 33: The system of any example herein, in particular example 30, wherein the cap has a disc shape.

Example 34: The system of any example herein, in particular example 30, wherein the cap comprises an aperture at a central portion of the cap.

Example 35: The system of any example herein, in particular example 30, wherein the actuation wire extends through a length of the cap.

Example 36: The system of any example herein, in particular example 1, further comprising an actuation wire extending at least partially through a lumen of the valve and a covering at least partially enclosing the actuation wire.

Example 37: The system of any example herein, in particular example 36, wherein the actuation wire has a coiled form.

Example 38: The system of any example herein, in particular example 37, wherein the actuation wire is configured to have a stretched default form, and wherein the actuation wire is configured to compress in response to a current.

Example 39: The system of any example herein, in particular example 38, wherein the covering is configured to expand laterally to increase blockage of the lumen in response to compression of the actuation wire.

Example 40: The system of any example herein, in particular example 1, wherein the valve further comprises an inner frame comprising a network of struts extending between arms of the inner frame, and wherein the arms of the inner frame have a generally straight default form.

Example 41: The system of any example herein, in particular example 40, wherein the arms of the inner frame are configured to compress to an hourglass form in response to a current at the valve.

Example 42: The system of any example herein, in particular example 1, further comprising one or more actuation wires coiled at least partially around the outer frame.

Example 43: The system of any example herein, in particular example 42, wherein the one or more actuation wires are configured to compress a midsection of the outer frame in response to a current through the one or more actuation wires.

Example 44: The system of any example herein, in particular example 1, further comprising one or more arms extending longitudinally along the outer frame and one or more actuation wires extending along the one or more arms.

Example 45: The system of any example herein, in particular example 44, wherein each of the one or more arms comprises an inward bulge.

Example 46: The system of any example herein, in particular example 44, wherein the one or more actuation wires are configured to cause the one or more arms to extend inwardly in response to a current through the one or more actuation wires.

Example 47: The system of any example herein, in particular example 1, wherein the valve further comprises an inner frame forming one or more curved pathways.

Example 48: The system of any example herein, in particular example 47, wherein the one or more curved pathways form a tesla valve.

Example 49: The system of any example herein, in particular example 47, wherein the valve comprises one or more stoppers configured to block blood flow between the outer frame and the inner frame to force blood flow through the inner frame in response to a current at the valve.

Example 50: The system of any example herein, in particular example 1, further comprising a ratcheting mechanism configured to control blockage of the valve between multiple positions.

Example 51: The system of any example herein, in particular example 1, wherein the valve comprises two or more leaflets and one or more latches configured to hold the two or more leaflets in predetermined positions.

Example 52: The system of any example herein, in particular example 51, wherein the one or more latches extend from the outer frame.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Delivery systems as described herein may be used to position catheter tips and/or catheters to various areas of a human heart. For example, a catheter tip and/or catheter may be configured to pass from the right atrium into the coronary sinus. However, it will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas. It will be understood that other types of medical implant devices and/or procedures can be delivered to the coronary sinus using a delivery system as described herein, including for example ablation procedures, drug delivery and/or placement of coronary sinus leads.

What is claimed is:

1. A system for regulating blood flow through a blood vessel of a heart, the system comprising:
a valve comprising an outer frame and two or more leaflets, the valve sized for placement at least partially within the blood vessel, the two or more leaflets sized to overlap or contact each other in an expanded state;
two or more actuation wires each coupled to different leaflets of the two or more leaflets to individually control movement of the two or more leaflets to the expanded state; and
a controller disposed outside the heart to transmit signals to the valve to activate the two or more actuation wires.

2. The system of claim 1, further comprising a first anchor tethered to the valve and extending proximally from the valve.

3. The system of claim 1, further comprising collapsible support arms extending along each of the two or more leaflets.

4. The system of claim 1, further comprising a balloon coupled to an inner surface of the outer frame, the balloon having a deflated default form and having a hollow interior to be inflated with a gas or fluid to at least partially occlude a lumen of the outer frame.

5. The system of claim 1, further comprising:
a cap disposed within a lumen of the valve; and
an actuation wire coupled to the cap.

6. The system of claim 1, wherein the valve further comprises an inner frame comprising a network of struts extending between arms of the inner frame, and wherein the arms of the inner frame have a generally straight default form.

7. The system of claim 1, further comprising:
one or more arms extending longitudinally along the outer frame; and
one or more actuation wires extending along the one or more arms.

8. The system of claim 1, wherein the valve further comprises an inner frame forming one or more curved pathways.

9. The system of claim 1, further comprising a ratcheting mechanism to control blockage of the valve between multiple positions.

10. The system of claim 1, wherein the valve comprises one or more latches to hold the two or more leaflets in predetermined positions.

11. The system of claim 1, further comprising a sensor to transmit blood flow signals to the controller.

12. A system for regulating blood flow through a blood vessel of a heart, the system comprising:
a valve comprising an outer frame and a cap sized to at least partially occlude a lumen of the outer frame, the valve sized for placement at least partially within the blood vessel; and
a controller disposed outside the heart to transmit signals to the valve.

13. The system of claim 12, further comprising:
one or more leaflets extending across a lumen of the outer frame; and
collapsible support arms extending along each of the one or more leaflets.

14. The system of claim 12, further comprising one or more actuation wires coupled to the valve to control movement of the cap.

15. The system of claim 12, further comprising a coil coupled to the cap, the coil being biased to hold the cap away from the outer frame.

16. The system of claim 15, wherein the controller sends signals to the coil to cause the coil to compress and move the cap towards the outer frame and occlude the lumen of the outer frame.

17. The system of claim 12, wherein the cap has a circular form sized to cover an end of the outer frame.

18. A system for regulating blood flow through a blood vessel of a heart, the system comprising:
a valve comprising an outer frame and one or more occluders, the valve sized for placement at least partially within the blood vessel, the one or more occluders coupled to one or more actuators, the outer frame comprising one or more latches to hold the one or more occluders in predetermined positions;
a controller disposed outside the heart to transmit signals to the one or more actuators to cause movement of the one or more occluders relative to the outer frame; and
a sensor coupled to the valve to transmit blood flow signals to the controller.

19. The system of claim 18, wherein the one or more actuators are each coupled to different occluders of the one or more occluders to individually control movement of the one or more occluders.

20. The system of claim 18, wherein the one or more occluders are sized to overlap or contact each other in an expanded state.
